(19) United States
(12) Patent Application Publication
(10) Pub. No.: US 2005/0019955 A1
(43) Pub. Date: Jan. 27, 2005

(54) Luminescent heterodiamondoids as biological labels

(76) Inventors: Jeremy E. Dahl, Palo Alto, CA (US); Robert M. Carlson, Petaluma, CA (US); Shenggao Liu, Hercules, CA (US); Wasiq Bokhari, Menlo Park, CA (US)

Correspondence Address: BURNS DOANE SWECKER & MATHIS L L P, POST OFFICE BOX 1404, ALEXANDRIA, VA, 22313-1404, US

(21) Appl. No.: 10/758,679

(22) Filed: Jan. 15, 2004

Related U.S. Application Data
(60) Provisional application No. 60/489,550, filed on Jul. 23, 2003.

(51) Int. Cl. G01N 33/551

(52) U.S. Cl. 436/524

(57) ABSTRACT

Novel biological labels are disclosed herein. The label comprises a functionalized heterodiamondoid functioning as a biological probe, the probe having an affinity for a target analyte molecule. Upon absorption of incident excitation radiation, color center(s) located within the heterodiamondoid are caused to luminesce. The photoemitted light may be detected and analyzed to yield information about the analyte. The color centers in the heterodiamondoid will typically comprise nitrogen-vacancy and/or nitrogen-pore complexes, but may also comprise a dopant impurity atom such as a rare earth or transition metal element.

### REFERENCE TO RELATED APPLICATIONS

The present application claims the benefit of U.S. Provisional Patent Application No. 60/489,550 filed Jul. 23, 2003. U.S. Provisional Patent Application No. 60/489,550 is hereby incorporated by reference in its entirety.

### BACKGROUND OF THE INVENTION

1. Field of the Invention

Embodiments of the present invention are directed in general toward the uses of heterodiamondoids as labels for use in biological systems. Specifically, functionalized heterodiamondoids may function as labels in probes capable of binding to a biological target of interest (the analyte) whereupon the probe-target complex, termed a biological label, is capable of luminescence when exposed to an energy source.

2. State of the Art

Fluorescent labeling of biological systems is a well known analytical tool used in biotechnology and analytical chemistry. Applications for such fluorescent labeling include fluorescence microscopy, histology, flow cytometry, florescence in-situ hybridization, DNA sequencing, immunoassays, binding assays, and separation procedures. Conventionally, fluorescent labeling involves the use of an organic dye molecule which is bonded to a moiety that in turn can be conjugated to a particular biological system. The presence of the conjugated organic dye is then identified by excitation of the dye molecule to cause it to fluoresce.

There are a number of problems with such conventional systems. One is that the emission of light in the visible region from an excited dye molecule is usually characterized by the presence of a broad emission spectrum. As a result, there is a severe limitation on the number of different dye molecules which may be used either simultaneously or sequentially in an analysis since it is difficult to discriminate individual substances as a result of the broad spectrum. Another problem is that most dye molecules have a relatively narrow absorption spectrum, thus requiring either multiple excitation beams (used either in tandem or sequentially for multiple wavelength probes), or else a broad spectrum excitation source (which is sequentially used with different filters for sequential excitation of a series of probes respectively excited at different wavelengths).

A third problem frequently encountered with existing dye molecule labels is that of photostability. Available fluorescent molecules bleach, or irreversibly cease to emit light under repeated cycles of absorption and emission. In addition, the molecular probes used for the study of systems by electron microscopy techniques are completely different from probes used for study by fluroescence. Thus, it is not possible to label a material with a single type of probe for both electron microscopy and for fluorescence.

Another approach that has been taken for the detection of biomolecules using various assays has been conductor nanocrystals, or “quantum dots,” which are known in the art. Examples of quantum dots known in the art have a core material that typically comprises CdSe, CdS, and CdTe, collectively known as CdX. CdX quantum dots are usually passivated with an inorganic coating, called a “shell.” Passivating the surface of the core quantum dot can result in an increase in the quantum yield of the luminescence emission, depending on the nature of the inorganic coating. The shell which is typically used to passivate on the quantum dot may be represented by the formula YZ, where Y is Cd or Zn, and Z is S or Se. Quantum dots having a CdX core and a YZ shell have been described in the art. To make quantum dots useful in biological applications, it is desirable that the quantum dots are water-soluble.

Diamondoids are known in the art. Elemental carbon has the electronic structure 1s²2s²2p², where the outer shell 2s and 2p electrons have the ability to hybridize according to two different schemes. The so-called sp³ hybridization comprises four identical σ bonds arranged in a tetrahedral manner. The so-called sp²-hybridization comprises three trigonal (as well as planar) σ bonds with an unhybridized p-electron occupying a π orbital in a bond oriented perpendicular to the plane of the σ bonds. At the “extremes” of crystalline morphology are diamond and graphite. In diamond, the carbon atoms are tetrahedrally bonded with sp³-hybridization. Graphite comprises planar “sheets” of sp²-hybridized atoms, where the sheets interact weakly through perpendicularly oriented π bonds. Carbon exists in other morphologies as well, including amorphous forms called “diamond-like carbon” (DLC), and the highly symmetrical spherical and rod-shaped structures called “fullerenes” and “nanotubes,” respectively.

Diamond is an exceptional material because it scores highest (or lowest, depending on one's point of view) in a number of different categories of properties. Not only is it the hardest material known, but it has the highest thermal conductivity of any material at room temperature. It displays superb optical transparency from the infrared through the ultraviolet, has the highest refractive index of any clear material, and is an excellent electrical insulator because of its very wide bandgap. It also displays high electrical breakdown strength, and very high electron and hole mobilities.

A form of carbon not discussed extensively in the literature is the “diamondoid.” Diamondoids are bridged-ring cycloalkanes that comprise adamantane, diamantane, triamantane, and the tetramers, pentamers, hexamers, heptamers, octamers, nonamers, decamers, etc., of adamantane (tricyclo[3.3.1.1^{3,7}] decane), adamantane having the stoichiometric formula C₁₀H₁₆, in which various adamantane units are face-fused to form larger structures. These adamantane units are essentially subunits of diamondoids. The compounds have a “diamondoid” topology in that their carbon atom arrangements are superimposable on a fragment of an FCC (face centered cubic) diamond lattice. According to embodiments of the present invention, electron donating and withdrawing heteroatoms may be inserted into the diamond lattice, thereby creating an n and p-type (respectively) material. The heteroatom is essentially an impurity atom that has been “folded into” the diamond lattice, and thus many of the disadvantages of the prior art methods have been avoided. A diamondoid containing one or more heteroatoms may be termed a “heterodiamondoid.”

Additionally, these materials may be derivatized such that functional groups are attached as pendant groups to the diamondoid molecule. Functionalized diamondoids are capable of undergoing further reactions, such as polymerizations. As reported herein, functional groups may also enter into specific reactions to bind with biological analytes and the like.

It is therefore desirable to provide a stable fluorophore material for biological applications having a wide absorption spectrum, while also capable of providing a detectable signal in response to exposure to energy, without the presence of the large emission tails characteristic of current dye molecules. It would be equally desirable to provide a single, stable probe material which can be used to image a sample by both light and electron microscopy.

There is also a need for heterodiamondoid nanocrystals which are water soluble, and functionalized to enhance stability in aqueous solutions. It is desirable that the fluorophores used in a biological probe can be excited with a single wavelength of light resulting in detectable luminescence emissions of high quantum yield and with discrete luminescent peaks. It is desirable that the biological probe be stable in aqueous settings, and capable of binding ligands, molecules, or analytes of various types. Additional advantages include the biocompatibility of diamond with biological materials.

### SUMMARY OF THE INVENTION

Embodiments of the present invention are directed toward novel fluorescent labels based on heterodiamondoids. Conventional labeling techniques have relied on fluorescing organic dyes, but there are a number of problems with such analytical systems. One is that the emission of light in the visible region from an excited dye molecule is usually characterized by the presence of a broad emission spectrum. Another problem is that most dye molecules have a relatively narrow absorption spectrum, thus requiring multiple excitation beams. A third problem is that of photostability, where conventional fluorescent molecules have the tendency to bleach, or irreversibly cease to emit light under repeated cycles of absorption and emission.

The present embodiments include an overall biological label system which may comprise a fluorescent diamondoid-containing probe, a light source for delivering energy to the biological label, and a detection system for processing the light emitted from the biological label. The biological probe may comprise a diamondoid or diamondoid-containing material with at least one color center. The color center may comprise at least one nitrogen-containing heteroatom in a heterodiamondoid, where the heteroatom may be positioned adjacent to at least one vacancy or pore. In one mode of operation, the probe is introduced into an environment containing the biological target and the probe associates with the target via a specific reaction with a functional group on the probe such as hybridization or the like. The probe/target complex may be spectroscopically viewed by radiation of the complex with an excitation light source. Of course, the complex may be spectroscopically excited by other forms of excitation, such as electrical, chemical, thermal, or tribological excitation. The labeled probe/target complex emits a characteristic spectrum which can be observed and measured.

According to embodiments of the present invention, the functional groups of the heterodiamondoid probe allow the heterodiamondoid to physically interact with the biological molecules of interest (i.e., the targets). Without limiting the scope of the invention, the functional groups of the heterodiamondoids can bind to proteins, nucleic acids, cells, subcellular organelles, lipids, carbohydrates, antigens, antibodies, nucleic acids, and other biological molecules. The affinity between the functional groups of the heterodiamondoid probe and the target molecule (hereinafter referred to as target analyte or simply analyte) may be based upon any of a different number of binding schemes or associations, including but not limited to van der Waals attractions, hydrophilic attractions, hydrophobic attractions, ionic and/or covalent bonding, electrostatic, and/or magnetic attractions.

In one embodiment of the present invention, a biological label is provided that comprises at least one luminescent color center, the color center comprising a nitrogen heteroatom substitutionally positioned on a diamondoid lattice site adjacent to at least one vacancy or pore. In another embodiment of the present invention, a biological label comprising at least one optically active dopant inserted into a diamondoid-containing material. In these embodiments, the diamondoid is a lower diamondoid selected from the group consisting of adamantane, diamantane, and triamantane, and heterodiamondoid derivatives thereof. The diamondoid may also comprise a higher diamondoid selected from the group consisting of tetramantane, pentamantane, hexamantane, heptamantane, octamantane, nonamantane, decamantane, and undecamantane, and heterodiamondoid derivatives thereof.

In yet another embodiment of the present invention is a method of detecting a target analyte, the method comprising the steps of:
a) providing a heterodiamondoid-containing probe;
b) binding the heterodiamondoid-containing probe to the target analyte, thus creating a biological label;
c) exciting the biological label with energy such that the biological label is caused to luminesce; and
d) detecting light emitted from the excited biological label.

The present methods may further include the step of passing the biological label through a cell membrane after the heterodiamondoid-containing probe is bound to the target analyte, or the step of passing the heterodiamondoid-containing probe through a cell membrane, and then reacting the heterodiamondoid-containing probe with the target analyte.

### BRIEF DESCRIPTION OF THE DRAWINGS

FIG. 1 is an overview of the general subject of the present invention, showing the steps of isolating diamondoids from petroleum, synthesizing a functionalized heterodiamondoid probe, binding the probe with a target analyte to produce a labeled analyte, and causing the labeled analyte to luminesce;

FIG. 2 shows an exemplary process flow for isolating diamondoids from petroleum;

FIG. 3 illustrates the relationship of a diamondoid to the diamond crystal lattice, and enumerates by stoichiometric formula many of the diamondoids that are available;

FIG. 4 illustrates exemplary lattice positions where a heteroatom may be substitutionally positioned;

FIGS. 5A-B illustrate exemplary pathways for synthetically producing heterodiamondoids;

FIG. 6 illustrates an exemplary tetramer of heterodiamondoids that may comprise the biological probe;

FIG. 7 is an stereogram illustrating how an exemplary diamondoid, [1(2,3)4] pentamantane, packs to form a molecular crystal that may comprise the biological probe;

FIG. 8 is a chart defining the terminology used to describe nitrogen heteroatoms in diamond (from I. Kiflawi et al. in “Theory of aggregation of nitrogen in diamond,” Properties, Growth and Applications of Diamond, edited by M. H. Nazaré and A. J. Neves (Inspec, London, 2001), pp. 130-133);

FIG. 9 shows various configurations of substitutionally positioned nitrogen atoms and vacancies in diamond that lead to photoluminescent color centers (from R. Jones et al. in “Theory of aggregation of nitrogen in diamond” in Properties, Growth and Applications of Diamond, edited by M. H. Nazaré and A. J. Neves (Inspec, London, 2001), pp. 127-129);

FIGS. 10A-B are exemplary diamondoid-containing materials contemplated to have photoluminescent nitrogen-vacancy color centers;

FIGS. 11A-B are exemplary diamondoid-containing materials that include a dopant atom for creating a photoemissive event; and

FIG. 12 illustrates an exemplary operational use of the biological labels contemplated by the present invention.

### DETAILED DESCRIPTION OF THE INVENTION

Biolabels comprising heterodiamondoid-containing materials may enable the creation of novel biolabels with unique attributes, particularly with regard to size, shape, ease of functionalization, and the fact that they have a precisely determined structure. Since most higher diamondoids are between 1-2 nm in size, the advantages of using them in biolabels relative to conventional materials are that they are potentially smaller than other nanoparticle based labels such as quantum dots or metal nanospheres. Smaller size enables higher diamondoid based biolabels to find more versatile uses in research by enhancing their bio-intake as well as allowing them to bind to smaller bio-molecules. The fact that the luminescing heterodiamondoid-containing materials of the present biolables display different shapes enables the creation of shape-specific biolabels for various purposes. In addition, docking or un-docking events of the biolabels may change their fluorescence characteristics and serve as useful indicators for cellular mechanisms.

Ease of functionalization of the present heterodiamondoids is an especially attractive feature, particularly in view of the difficulty in the art of bioconjugating the well known quantum dots. The difficulty of bioconjugating quantum dots can potentially restrict their usage. With ease of functionalization, higher diamondoid based biolabels may be bioconjugated for a potentially much larger set of cellular events and regulators. Additionally, the precisely determined structure of the present heterodiamondoids is advantageous because higher diamondoids are individual molecules and their structures are completely known, unlike nanoparticles like quantum dots or nanospheres. The knowledge of the precise structure and properties of the diamondoid molecules enables the creation of highly specific labels.

Nanoparticle based biolabels have the advantage of robust emission characteristics over dye based labels because they do not suffer from photo-bleaching. In contrast, dye based labels are experimentally easier and more versatile because of simpler chemistry. Higher diamondoid based biolabels potentially combine performance robustness of nanoparticles with the experimental simplicity of dye chemistry.

The color centers of the present biolabels are contemplated to have luminescent properties. Luminescence has been generally defined by M. Fox in Optical Properties of Solids (Oxford University Press, New York, 2001), p. 2, as a general name given to the process of spontaneous emission of light by excited atoms in a solid-state material. The atoms of the material may be raised to an excited state prior to spontaneous emission via a number of different mechanisms, one of which being the absorption of light. Luminescence can thus accompany the propagation of light in an absorbing medium. The light is emitted in all directions, and the emitted light has a different frequency than that of the incoming light.

Fox goes on to point out that luminescence does not always have to accompany absorption. Since a characteristic amount of time is required for the excited atoms to re-emit light by spontaneous emission, it can be possible for the excited atoms to dissipate the excitation as heat before the radiative emission process has an opportunity to occur. The efficiency of luminescence, therefore, is intimately related to the nature of materials and systems whose luminescence is desired.

Photoluminescence is a term generally reserved to describe a phenomenon wherein the fluorescence event is caused by an incident beam of photons (“excitation radiation”). In contrast, electroluminescence describes a similar fluorescent event, but in this case, the event is caused by electron beam excitation. The fluorescent event may be caused by other types of input energy. For example, if the form of the injected energy is due to thermal means, such as the application of heat, then the appropriate term is thermoluminescence. The application of chemical energy leads to chemiluminescence. An energy input that results from the frictional contact between two substances is termed triboluminescence. Each of these types of energy input that result in a fluorescence event are contemplated by embodiments of the present invention.

The present disclosure will be organized in the following manner: first, a description of how diamondoids may be isolated, functionalized, and chemically altered to provide functionalized heterodiamondoids is provided. Following that is a description of the binding chemistry; in other words, how the functionalized heterodiamondoid (the biological probe) may be reacted with a target analyte, the substance or species whose presence, location, distribution, and other such information is desired to be known. The analyte is now “labeled.” Transport of the functionalized diamondoid (before reaction with the target molecule), and transport of the labeled analyte (after reaction with the target molecule) is discussed. The labeled analyte (functionalized heterodiamondoid probe and target analyte complex) may then be excited with energy to generate a luminescent event. Systems and methods may be provided for detecting the emitted light, and detection systems are discussed briefly.

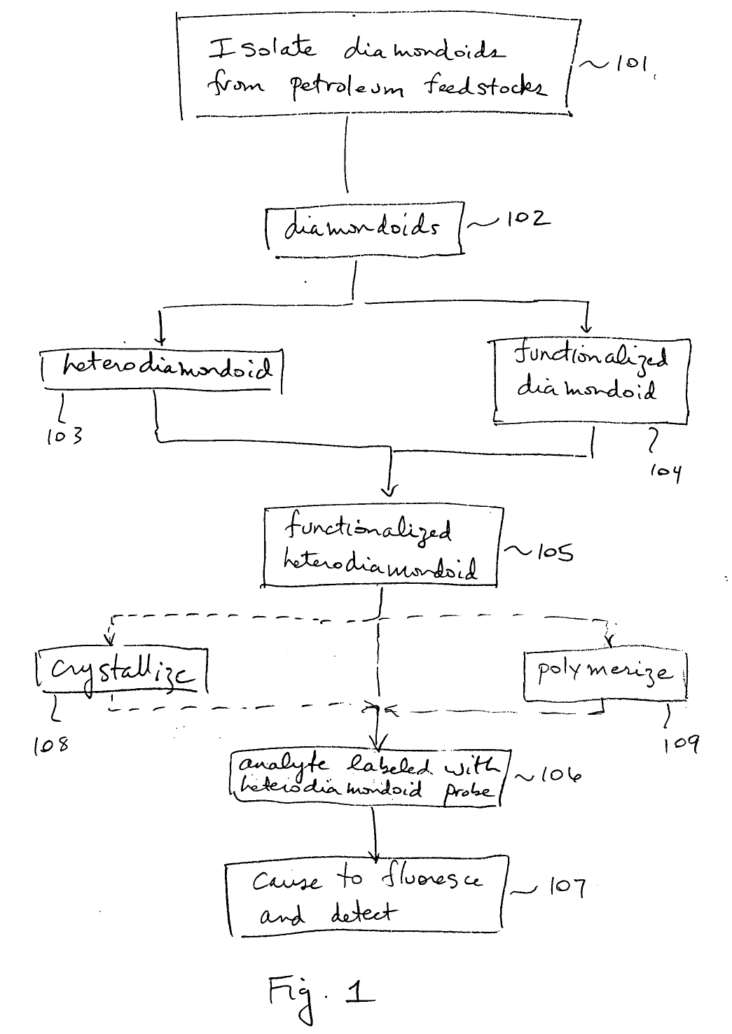

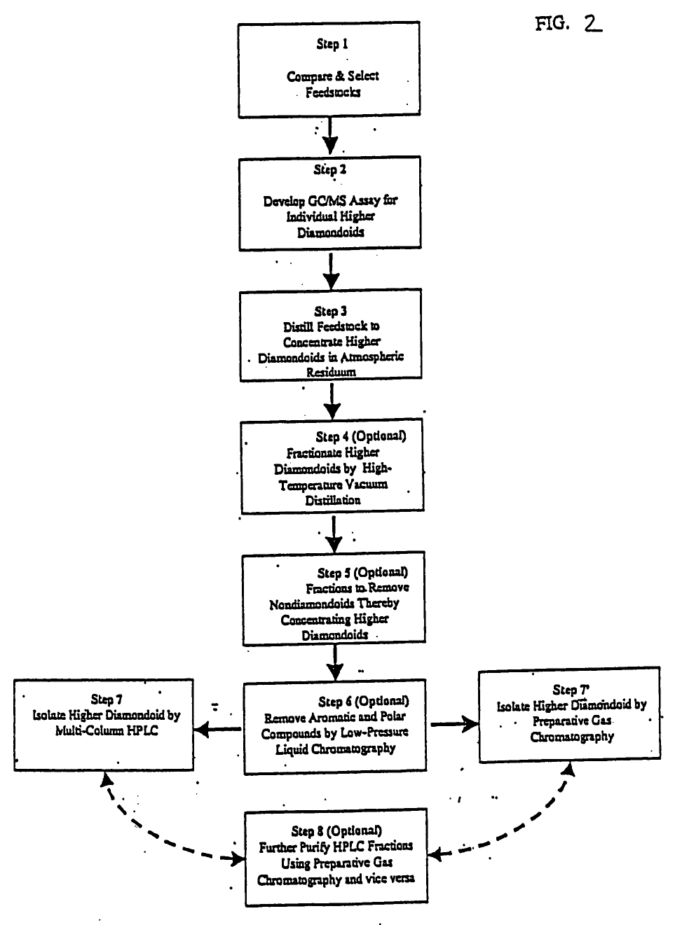

An overview of the embodiments of the present invention is shown in FIG. 1. Referring to FIG. 1, diamondoids are isolated from a petroleum feedstock in a step 101, producing diamondoids 102. The following sequence of steps produce a functionalized heterodiamondoid 105, and there are at least two possible routes to accomplish this goal. In one embodiment, a heteroatom (which may be nitrogen) is inserted into a carbon atom lattice site of the diamondoid 102, thus producing heterodiamondoid 103. A functional group may then be attached to the heterodiamondoid 103 to produce the functionalized heterodiamondoid 105. Alternatively, the diamondoid 102 may first be reacted with a functional group to produce functionalized diamondoid 104, and then a heteroatom (which again may be nitrogen) is inserted into a lattice site to produce the functionalized heterodiamondoid 105. The purpose of generating the substitutionally positioned heteroatom is to create a photoluminescent color center, and the purpose of functionalizing the diamondoid 102 is to provide a means by which the diamondoid 102 may attach to the biological compound (analyte) whose presence is to be determined and/or measured.

Thus, the functionalized heterodiamondoid 105 may be reacted with an analyte in a step 106 to produce the analyte labeled with heterodiamondoid probe, which may then be energized to an excited state in a step 107 such that photoemission can occur. In an alternative embodiment, the functionalized heterodiamondoid 105 may be crystallized in a step 108 to create a larger species for reaction with analyte than an individual heterodiamondoid would have provided. Additionally, the functionalized heterodiamondoid 105 may be polymerized in a step 109 to create a larger species for reaction with analyte.

Definition of Diamondoids

The term “diamondoids” refers to substituted and unsubstituted caged compounds of the adamantane series including adamantane, diamantane, triamantane, tetramantane, pentamantane, hexamantane, heptamantane, octamantane, nonamantane, decamantane, undecamantane, and the like, including all isomers and stereoisomers thereof. The compounds have a “diamondoid” topology, which means their carbon atom arrangement is superimposable on a fragment of an FCC diamond lattice. Substituted diamondoids comprise from 1 to 10 and preferably 1 to 4 independently-selected alkyl substituents.

Adamantane chemistry has been reviewed by Fort, Jr. et al. in “Adamantane: Consequences of the Diamondoid Structure,” Chem. Rev. vol. 64, pp. 277-300 (1964). Adamantane is the smallest member of the diamondoid series and may be thought of as a single cage crystalline subunit. Diamantane contains two subunits, triamantane three, tetramantane four, and so on. While there is only one isomeric form of adamantane, diamantane, and triamantane, there are four different isomers of tetramantane (two of which represent an enantiomeric pair), i.e., four different possible ways of arranging the four adamantane subunits. The number of possible isomers increases non-linearly with each higher member of the diamondoid series, pentamantane, hexamantane, heptamantane, octamantane, nonamantane, decamantane, etc.

Adamantane, which is commercially available, has been studied extensively. The studies have been directed toward a number of areas, such as thermodynamic stability, functionalization, and the properties of adamantane-containing materials. For instance, the following patents discuss materials comprising adamantane subunits: U.S. Pat. No. 3,457,318 teaches the preparation of polymers from alkenyl adamantanes; U.S. Pat. No. 3,832,332 teaches a polyamide polymer forms from alkyladamantane diamine; U.S. Pat. No. 5,017,734 discusses the formation of thermally stable resins from adamantane derivatives; and U.S. Pat. No. 6,235,851 reports the synthesis and polymerization of a variety of adamantane derivatives.

In contrast, the diamondoids tetramantane and higher (known as “higher” diamondoids) have received comparatively little attention in the scientific literature. McKervay et al. have reported the synthesis of anti-tetramantane in low yields using a laborious, multistep process in “Synthetic Approaches to Large Diamondoid Hydrocarbons,” Tetrahedron, vol. 36, pp. 971-992 (1980). To the inventors' knowledge, this is the only higher diamondoid that has been synthesized to date. Lin et al. have suggested the existence of, but did not isolate, tetramantane, pentamantane, and hexamantane in deep petroleum reservoirs in light of mass spectroscopic studies, reported in “Natural Occurrence of Tetramantane (C₂₂H₂₈), Pentamantane (C₂₆H₃₂) and Hexamantane (C₃₀H₃₆) in a Deep Petroleum Reservoir,” Fuel, vol. 74(10), pp. 1512-1521 (1995). The possible presence of tetramantane and pentamantane in pot material after a distillation of a diamondoid-containing feedstock has been discussed by Chen et al. in U.S. Pat. No. 5,414,189.

The four tetramantane structures are iso-tetramantane [1(2)3], anti-tetramantane [121] and two enantiomers of skew-tetramantane [123], with the bracketed nomenclature for these diamondoids in accordance with a convention established by Balaban et al. in “Systematic Classification and Nomenclature of Diamond Hydrocarbons-I,” Tetrahedron vol. 34, pp. 3599-3606 (1978). All four tetramantanes have the formula C₂₂H₂₈ (molecular weight 292). There are ten possible pentamantanes, nine having the molecular formula C₂₆H₃₂ (molecular weight 344) and among these nine, there are three pairs of enantiomers represented generally by [12(1)3], [1234], [1213] with the nine enantiomeric pentamantanes represented by [12(3)4], [1(2,3)4], [1212]. There also exists a pentamantane [1231] represented by the molecular formula C₂₅H₃₀ (molecular weight 330).

Hexamantanes exist in thirty nine possible structures with twenty eight having the molecular formula C₃₀H₃₆ (molecular weight 396) and of these, six are symmetrical; ten hexamantanes have the molecular formula C₂₉H₃₄ (molecular weight 382) and the remaining hexamantane [12312] has the molecular formula C₂₆H₃₀ (molecular weight 342).

Heptamantanes are postulated to exist in 160 possible structures with 85 having the molecular formula C₃₄H₄₀ (molecular weight 448) and of these, seven are achiral, having no enantiomers. Of the remaining heptamantanes 67 have the molecular formula C₃₃H₃₈ (molecular weight 434), six have the molecular formula C₃₂H₃₆ (molecular weight 420) and the remaining two have the molecular formula C₃₀H₃₄ (molecular weight 394).

Octamantanes possess eight of the adamantane subunits and exist with five different molecular weights. Among the octamantanes, 18 have the molecular formula C₃₄H₃₈ (molecular weight 446). Octamantanes also have the molecular formula C₃₈H₄₄ (molecular weight 500); C₃₇H₄₂ (molecular weight 486); C₃₆H₄₀ (molecular weight 472), and C₃₃H₃₆ (molecular weight 432).

Nonamantanes exist within six families of different molecular weights having the following molecular formulas: C₄₂H₄₈ (molecular weight 552), C₄₁H₄₆ (molecular weight 538), C₄₀H₄₄ (molecular weight 524, C₃₈H₄₂ (molecular weight 498), C₃₇H₄₀ (molecular weight 484) and C₃₄H₃₆ (molecular weight 444).

Decamantane exists within families of seven different molecular weights. Among the decamantanes, there is a single decamantane having the molecular formula C₃₅H₃₆ (molecular weight 456) which is structurally compact in relation to the other decamantanes. The other decamantane families have the molecular formulas: C₄₆H₅₂ (molecular weight 604); C₄₅H₅₀ (molecular weight 590); C₄₄H₄₈ (molecular weight 576); C₄₂H₄₆ (molecular weight 550); C₄₁H₄₄ (molecular weight 536); and C₃₈H₄₀ (molecular weight 496).

Undecamantane exists within families of eight different molecular weights. Among the undecamantanes there are two undecamantanes having the molecular formula C₃₉H₄₀ (molecular weight 508) which are structurally compact in relation to the other undecamantanes. The other undecamantane families have the molecular formulas C₄₁H₄₂ (molecular weight 534); C₄₂H₄₄ (molecular weight 548); C₄₅H₄₈ (molecular weight 588); C₄₆H₅₀ (molecular weight 602); C₄₈H₅₂ (molecular weight 628); C₄₉H₅₄ (molecular weight 642); and C₅₀H₅₆ (molecular weight 656).

Isolation of Diamondoids from Petroleum Feedstocks

Feedstocks that contain recoverable amounts of higher diamondoids include, for example, natural gas condensates and refinery streams resulting from cracking, distillation, coking processes, and the like. Particularly preferred feedstocks originate from the Norphlet Formation in the Gulf of Mexico and the LeDuc Formation in Canada.

These feedstocks contain large proportions of lower diamondoids (often as much as about two thirds) and lower but significant amounts of higher diamondoids (often as much as about 0.3 to 0.5 percent by weight). The processing of such feedstocks to remove non-diamondoids and to separate higher and lower diamondoids (if desired) can be carried out using, by way of example only, size separation techniques such as membranes, molecular sieves, etc., evaporation and thermal separators either under normal or reduced pressures, extractors, electrostatic separators, crystallization, chromatography, well head separators, and the like.

A preferred separation method typically includes distillation of the feedstock. This can remove low-boiling, non-diamondoid components. It can also remove or separate out lower and higher diamondoid components having a boiling point less than that of the higher diamondoid(s) selected for isolation. In either instance, the lower cuts will be enriched in lower diamondoids and low boiling point non-diamondoid materials. Distillation can be operated to provide several cuts in the temperature range of interest to provide the initial isolation of the identified higher diamondoid. The cuts, which are enriched in higher diamondoids or the diamondoid of interest, are retained and may require further purification. Other methods for the removal of contaminants and further purification of an enriched diamondoid fraction can additionally include the following nonlimiting examples: size separation techniques, evaporation either under normal or reduced pressure, sublimation, crystallization, chromatography, well head separators, flash distillation, fixed and fluid bed reactors, reduced pressure, and the like.

The removal of non-diamondoids may also include a thermal treatment step either prior or subsequent to distillation. The thermal treatment step may include a hydrotreating step, a hydrocracking step, a hydroprocessing step, or a pyrolysis step. Thermal treatment is an effective method to remove hydrocarbonaceous, non-diamondoid components from the feedstock, and one embodiment of it, pyrolysis, is effected by heating the feedstock under vacuum conditions, or in an inert atmosphere, to a temperature of at least about 390° C., and most preferably to a temperature in the range of about 410 to 450° C. Pyrolysis is continued for a sufficient length of time, and at a sufficiently high temperature, to thermally degrade at least about 10 percent by weight of the non-diamondoid components that were in the feed material prior to pyrolysis. More preferably at least about 50 percent by weight, and even more preferably at least 90 percent by weight of the non-diamondoids are thermally degraded.

While pyrolysis is preferred in one embodiment, it is not always necessary to facilitate the recovery, isolation or purification of diamondoids. Other separation methods may allow for the concentration of diamondoids to be sufficiently high given certain feedstocks such that direct purification methods such as chromatography including preparative gas chromatography and high performance liquid chromatography, crystallization, fractional sublimation may be used to isolate diamondoids.

Even after distillation or pyrolysis/distillation, further purification of the material may be desired to provide selected diamondoids for use in the compositions employed in this invention. Such purification techniques include chromatography, crystallization, thermal diffusion techniques, zone refining, progressive recrystallization, size separation, and the like. For instance, in one process, the recovered feedstock is subjected to the following additional procedures: 1) gravity column chromatography using silver nitrate impregnated silica gel; 2) two-column preparative capillary gas chromatography to isolate diamondoids; 3) crystallization to provide crystals of the highly concentrated diamondoids.

An alternative process is to use single or multiple column liquid chromatography, including high performance liquid chromatography, to isolate the diamondoids of interest. As above, multiple columns with different selectivities may be used. Further processing using these methods allow for more refined separations which can lead to a substantially pure component.

Detailed methods for processing feedstocks to obtain higher diamondoid compositions are set forth in U.S. Provisional Patent Application No. 60/262,842 filed Jan. 19, 2001; U.S. Provisional Patent Application No. 60/300,148 filed Jun. 21, 2001; and U.S. Provisional Patent Application No. 60/307,063 filed Jul. 20, 2001, and a co-pending application titled “Processes for concentrating higher diamondoids,” by B. Carlson et al., assigned to the assignee of the present application. These applications are herein incorporated by reference in their entirety.

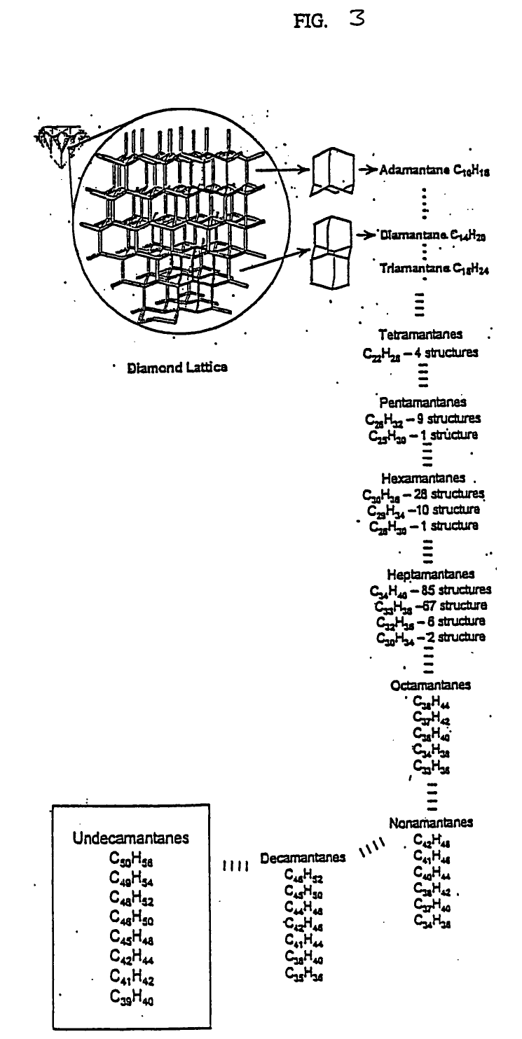

FIG. 2 shows a process flow illustrated in schematic form, wherein diamondoids may be extracted from petroleum feedstocks, and FIG. 3 enumerates the various diamondoid isomers that are available from embodiments of the present invention.

Synthesis of Heterodiamondoids

The term “heterodiamondoid” as used herein refers to a diamondoid that contains a heteroatom typically substitionally positioned on a lattice site of the diamond crystal structure. A heteroatom is an atom other than carbon, and according to present embodiments may be nitrogen, phosphorus, boron, aluminium, lithium, and arsenic. “Substitutionally positioned” means that the heteroatom has replaced a carbon host atom in the diamond lattice. Although most heteroatoms are substitutionally positioned, they may in some cases be found in interstitial sites as well.

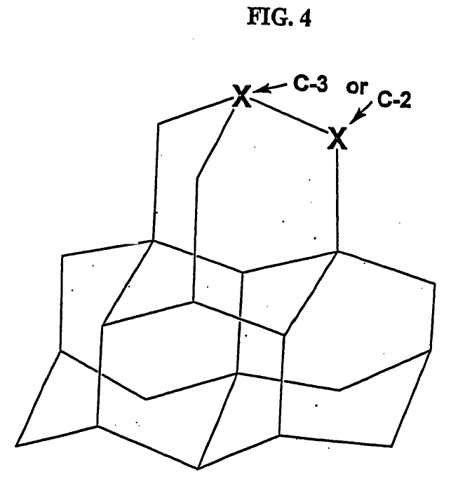

FIG. 4 illustrates exemplary heterodiamondoids, indicating the types of carbon positions where a heteroatom may be substitutionally positionned. These positions are labelled C-2 and C-3 in the exemplary diamonoid of FIG. 4. The term “diamondoid” will herein be used in a general sense to include diamondoids both with and without heteroatom substitutions. As disclosed above, the heteroatom may be an electron donating element such as N, P, or As, or a hole donating element such as B or Al. Emphasis in this disclosure will be placed on the nitrogen-containing heterodiamondoid, since it is the properties of the nitrogen-pore or nitrogen-vacancy color center that are being utilized in the present photoluminescent probes.

An exemplary synthesis of such heterodiamondoids will be discussed next. Although some heteroadamantane and heterodiamantane compounds have been synthesized in the past, and this may suggest a starting point for the synthesis of heterodiamondoids having more than two or three fused adamantane subunits, it will be appreciated by those skilled in the art that the complexity of the individual reactions and overall synthetic pathways increase as the number of adamantane subunits increases. For example, it may be necessary to employ protecting groups, or it may become more difficult to solubilize the reactants, or the reaction conditions may be vastly different from those that would have been used for the analagous reaction with adamantane. Nevertheless, it can be advantageous to discuss the chemistry underlying heterodiamondoid synthesis using adamantane or diamantane as a substrate because to the inventors' knowledge these are the only systems for which data has been available, prior to the present application.

Nitrogen hetero-adamantane compounds have been synthesized in the past. For example, in an article by T. Sasaki et al., “Synthesis of adamantane derivatives. 39. Synthesis and acidolysis of 2-azidoadamantanes. A facile route to 4-azahomoadamant-4-enes,” Heterocycles, Vol. 7, No. 1, p. 315 (1977). These authors reported a synthesis of 1-azidoadamantane and 3-hydroxy-4-azahomoadamantane from 1-hydroxyadamantane. The procedure consisted of a substitution of a hydroxyl group with an azide function via the formation of a carbocation, followed by acidolysis of the azide product.

In a related synthetic pathway, Sasaki et al. were able to subject an adamantanone to the conditions of a Schmidt reaction, producing a 4-keto-3-azahomoadamantane as a rearranged product. For details pertaining to the Schmidt reaction, see T. Sasaki et al., “Synthesis of Adamantane Derivatives. XII. The Schmidt Reaction of Adamantane-2-one,” J. Org. Chem., Vol. 35, No. 12, p. 4109 (1970).

Alternatively, an 1-hydroxy-2-azaadamantane may be synthesized from 1,3-dibromoadamantane, as reported by A. Gagneux et al. in “I-Substituted 2-heteroadamantanes,” Tetrahedron Letters No. 17, pp. 1365-1368 (1969). This was a multiple-step process, wherein first the di-bromo starting material was heated to a methyl ketone, which subsequently underwent ozonization to a diketone. The diketone was heated with four equivalents of hydroxylamine to produce a 1:1 mixture of cis and trans-dioximes; this mixture was hydrogenated to the compound 1-amino-2-azaadamantane dihydrochloride. Finally, nitrous acid transformed the dihydrochloride to the hetero-adamantane 1-hydroxy-2-azadamantane.

Alternatively, a 2-azaadamantane compound may be synthesized from a bicyclo[3.3.1]nonane-3,7-dione, as reported by J. G. Henkel and W. C. Faith, in “Neighboring group effects in the β-halo amines. Synthesis and solvolytic reactivity of the anti-4-substituted 2-azaadamantyl system,” in J. Org. Chem. Vol. 46, No. 24, pp. 4953-4959 (1981). The dione may be converted by reductive amination (although the use of ammonium acetate and sodium cyanoborohydride produced better yields) to an intermediate, which may be converted to another intermediate using thionyl choloride. Dehalogenation of this second intermediate to 2-azaadamantane was accomplished in good yield using LiAlH₄ in DME.

A synthetic pathway that is related in principal to one used in the present invention was reported by S. Eguchi et al. in “A novel route to the 2-aza-adamantyl system via photochemical ring contraction of epoxy 4-azahomoadamantanes,” J. Chem. Soc. Chem. Commun., p. 1147 (1984). In this approach, a 2-hydroxyadamantane was reacted with a NaN₃ based reagent system to form the azahomoadamantane, with was then oxidized by m-chloroperbenzoid acid (m-CPBA) to give an epoxy 4-azahomoadamantane. The epoxy was then irradiated in a photochemical ring contraction reaction to yield the N-acyl-2-aza-adamantane.

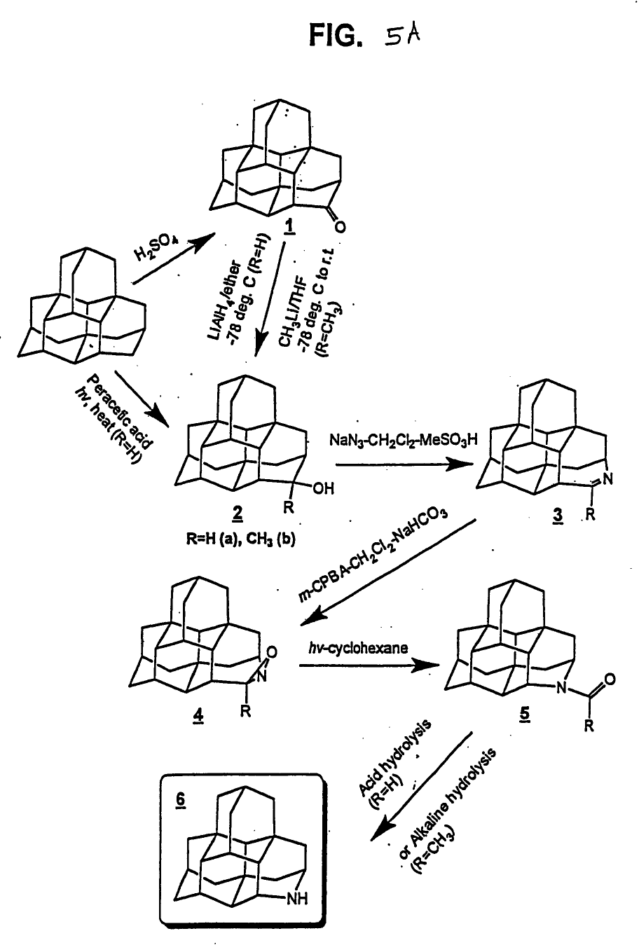

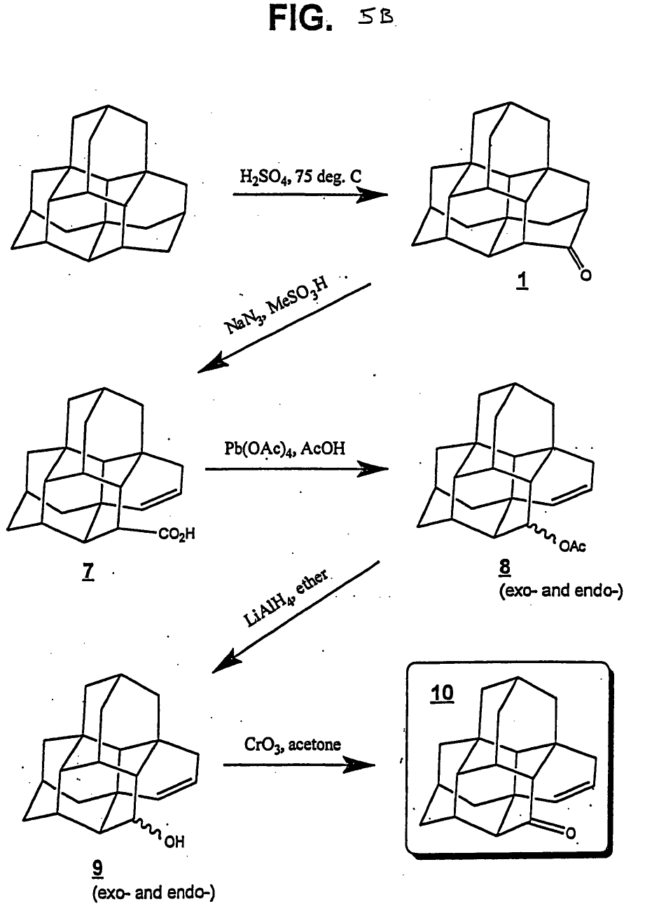

An exemplary reaction pathway for synthesizing a nitrogen-containing hetero iso-tetramantane is illustrated in FIG. 5A. It will be known to those of ordinary skill in the art that the reactions conditions of the pathway depicted in FIG. 5A will be substantially different from those of Eguchi due to the differences in size, solubility, and reactivities of tetramantane in relation to adamantane. A second pathway available for synthesizing nitrogen containing heterodiamondoids is illustrated in FIG. 5B.

In another embodiment of the present invention, a phosphorus-containing heterodiamondoid may be synthesized by adapting the pathway outlined by J. J. Meeuwissen et. al in “Synthesis of 1-phosphaadamantane,” Tetrahedron Vol. 39, No. 24, pp. 4225-4228 (1983). It is contemplated that such a pathway may be able to synthesize heterodiamondoids that contain both nitrogen and phosphorus atoms substitutionally positioned in the diamondoid structure, with the advantages of having two different types of electron-donating heteroatoms in the same structure.

After preparing a heterodiamondoid from a diamondoid having no impurity atoms contained therein, the resulting heterodiamondoid may be functionalized to generate a biological probe capable of binding to an analyte to form a labeled species. Alternatively, the diamondoid (having no impurity atoms) may be functionalized first, and then converted to the heteroatom form.

Further information on the synthesis of heterodiamondoids is provided in a U.S. patent application titled “Heterodiamondoids,” Ser. No. 10/622,130, filed Jul. 16, 2003, incorporated herein by reference in its entirety.

Functionalizing Heterodiamondoids

The heterodiamondoids discussed above may be derivatized (or functionalized) by attaching chemically active functional groups which in turn attach to a group capable of binding with a target analyte. The target analyte may in itself be capable with further reaction with another analyte. For example, the functional group on the heterodiamondoid may be capable of attaching the heterodiamondoid to an antigen, wherein the heterodiamondoid-antigen material may then be capable of a reaction with an antibody. Those skilled in the art will recognize that in this case the initial functional group of the heterodiamondoid behaves as (and could have been described as) a “linking agent” between the heterodiamondoid and the antigen.

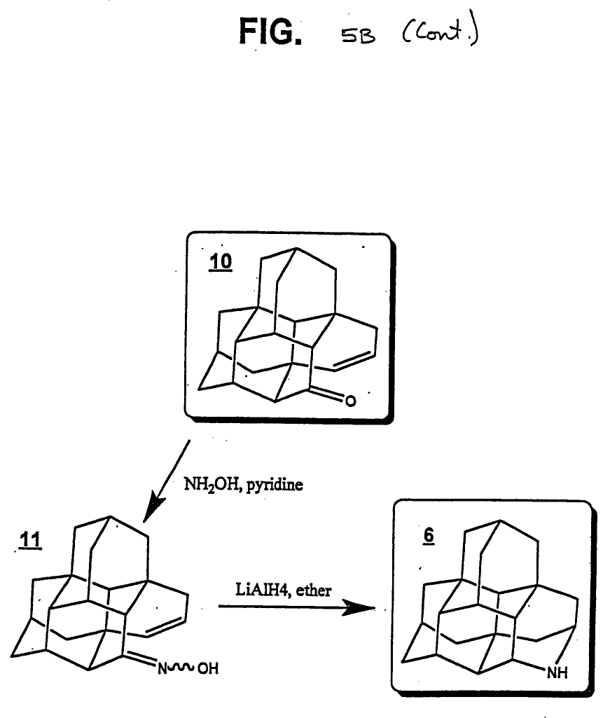

Alternatively, the attached functional groups may also be used to connect (or polymerize) several diamondoids together to construct a fluorolabel species prior to constructing the biological probe prior and reacting with an analyte. This covalently-linked complex of diamondoids may then be further functionalized to bond with a species capable of binding to a target analyte. This sequence of events is illustrated schematically in FIG. 6, in which a tetramer of heterodiamondoids has been prepared.

Referring to FIG. 6, heterodiamondoid 670 may be oxidized to diamondoid 671 having a carbonyl pendant group. In a step 672, two diamondoids 671 may be coupled to form the dimer 677. Likewise, two dimers 677 and 678 may be coupled to form the tetramer 679. This tetramer of diamondoids may then be functionalized for reaction with a species capable of binding a target analyte, or polymerized with other oligomers of diamondoids before undergoing further functionalization. Of course, it will be recognized by one skilled and art that the number of diamondoids comprising this oligomer (i.e., 4) was nearly exemplary, and a number of diamondoids may be used to construct the probe ranging from 1 to 100,000 or more. It is contemplated, however, that sizes of 1 to 100 diamondoids will be appropriate.

Functionalization of diamondoids, methods of forming diamondoid derivatives, and techniques for polymerizing derivatized diamondoids, have been previously discussed in U.S. patent application Ser. No. 60/334,939, entitled “Polymerizable Higher Diamondoid Derivatives,” by Shenggao Liu, Jeremy E. Dahl, and Robert M. Carlson, filed Dec. 4, 2001.

A derivatized diamondoid molecule has at least one functional group substituting one of the original hydrogens. As discussed in that application, there are two major reaction sequences that may be used to derivatize heterodiamondoids: nucleophilic (S_{N}1-type) and electrophilic (S_{E}2-type) substitution reactions.

S_{N}1-type reactions involve the generation of diamondoid carbocations, which subsequently react with various nucleophiles. Since tertiary (bridgehead) carbons of diamondoids are considerably more reactive than secondary carbons under S_{N}1 reaction conditions, substitution at a tertiary carbon is favored.

S_{E}2-type reactions involve an electrophilic substitution of a C—H bond via a five-coordinate carbocation intermediate. Of the two major reaction pathways that may be used for the functionalization of heterodiamondoids, the S_{N}1-type may be more widely utilized for generating a variety of heterodiamondoid derivatives. Mono and multi-brominated heterodiamondoids are some of the most versatile intermediates for functionalizing heterodiamondoids. These intermediates are used in, for example, the Koch-Haaf, Ritter, and Friedel-Crafts alkylation and arylation reactions. Although direct bromination of heterodiamondoids is favored at bridgehead (tertiary) carbons, brominated derivatives may be substituted at secondary carbons as well. For the latter case, when synthesis is generally desired at secondary carbons, a free radical scheme is often employed.

Although the reaction pathways described above may be preferred in some embodiments of the present invention, many other reaction pathways may certainly be used as well to functionalize a heterodiamondoid. These reaction sequences may be used to produce derivatized heterodiamondoids having a variety of functional groups, such that the derivatives may include heterodiamondoids that are halogenated with elements other than bromine, such as fluorine, alkylated diamondoids, nitrated diamondoids, hydroxylated diamondoids, carboxylated diamondoids, ethenylated diamondoids, and aminated diamondoids. See Table 2 of the co-pending application “Polymerizable Higher Diamondoid Derivatives” for a listing of exemplary substituents that may be attached to heterodiamondoids.

Diamondoids and heterodiamondoids, as well as derivatived forms thereof having substituents capable of entering into polymerizable reactions, may be subjected to suitable reaction conditions such that polymers are produced. The polymers may be homopolymers or heteropolymers, and the polymerizable diamondoid and/or heterodiamondoid derivatives may be co-polymerized with nondiamondoid, diamondoid, and/or heterodiamondoid-containing monomers. Polymerization is typically carried out using one of the following methods: free radical polymerization, cationic, or anionic polymerization, and polycondensation. Procedures for inducing free radical, cationic, anionic polymerizations, and polycondensation reactions are well known in the art.

Free radical polymerization may occur spontaneously upon the absorption of an adequate amount of heat, ultraviolet light, or high-energy radiation. Typically, however, this polymerization process is enhanced by small amounts of a free radical initiator, such as peroxides, aza compounds, Lewis acids, and organometallic reagents. Free radical polymerization may use either non-derivatized or derivatized heterodiamondoid monomers. As a result of the polymerization reaction a covalent bond is formed between diamondoid, nondiamondoid, and heterodiamondoid monomers such that the diamondoid or heterodiamondoid becomes part of the main chain of the polymer. In another embodiment, the functional groups comprising substituents on a diamondoid or heterodiamondoid may polymerize such that the diamondoids or heterodiamondids end up being attached to the main chain as side groups. Diamondoids and heterodiamonhdoids having more than one functional group are capable of cross-linking polymeric chains together.

For cationic polymerization, a cationic catalyst may be used to promote the reaction. Suitable catalysts are Lewis acid catalysts, such as boron trifluoride and aluminum trichloride. These polymerization reactions are usually conducted in solution at low-temperature.

In anionic polymerizations, the derivatized diamondoid or heterodiamdondoid monomers are typically subjected to a strong nucleophilic agent. Such nucleophiles include, but are not limited to, Grignard reagents and other organometallic compounds. Anionic polymerizations are often facilitated by the removal of water and oxygen from the reaction medium.

Polycondensation reactions occur when the functional group of one diamondoid or heterodiamondoid couples with the functional group of another; for example, an amine group of one diamondoid or heterodiamondoid reacting with a carboxylic acid group of another, forming an amide linkage. In other words, one diamondoid or heterodiamondoid may condense with another when the functional group of the first is a suitable nucleophile such as an alcohol, amine, or thiol group, and the functional group of the second is a suitable electrophile such as a carboxylic acid or epoxide group. Examples of heterodiamondoid-containing polymers that may be formed via polycondensation reactions include polyesters, polyamides, and polyethers.

Further information on the functionalization of diamondoids is provided in a U.S. patent application titled “Functionalized Higher Diamondoids,” Ser. No. 10/313,804, filed Dec. 6, 2002, incorporated herein by reference in its entirety.

Molecular Crystals

Diamondoids may crystallized into a solid, where the individual diamondoids comprising the solid are held together by Van der Waals forces (also called London or dispersive forces). Molecules that are held together in such a fashion have been discussed by J. S. Moore and S. Lee in “Crafting Molecular Based Solids,” Chemistry and Industry, July, 1994, pp. 556-559, and are called “molecular solids” in the art. These authors state that in contrast to extended solids or ionic crystals, the prefered arrangement of molecules in a molecular crystal is presumably one that minimizes total free energy, and thus the fabrication of a molecular crystal is controlled by thermodynamic considerations, unlike a synthetic process. An example of a molecular crystal comprising the pentamantane [1(2,3)4] will be discussed next.

In an exemplary embodiment, a molecular crystal comprisng [1(2,3)4] pentamantane was formed by the chromatographic and crystallographic techniques described above. These aggregations of diamondoids pack to form actual crystals in the sense that a lattice plus a basis may be defined. In this embodiment, the [1(2,3)4] pentamantane is found to pack in an orthorhombic crystal system having the space group Pnma, with unit cell dimensions a=11.4706, b=12.6418, and c=12.5169 angstroms, respectively. To obtain that diffraction data, a pentamantane crystal was tested in a Bruker SMART 1000 diffractometer using radiation of wavelength 0.71073 angstroms, the crystal maintained at a temperature of 90 K.

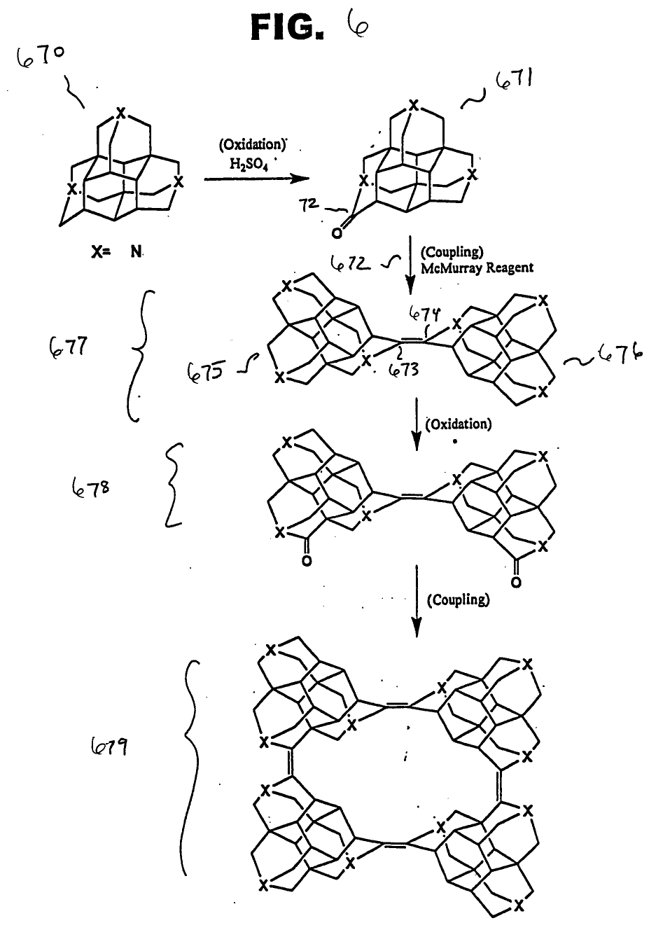

A unit cell of the pentamantane molecular crystal is illustrated in FIG. 7. This diagram illustrates the generalized manner in which heterodiamondoids may pack in order to be useful according to embodiments of the present invention. These molecular crystals display well-defined exterior crystal facets, and are transparent to visible radiation.

Referring to FIG. 7, the packing of the [1(2,3)4] pentamantane is illustrated in three dimensions by the stereogram having two images 702, 703, that may be viewed simultaneously. Each unit cell of the molecular crystal contains four pentamantane molecules, where the molecules are arranged such that there is one central cavity or pore 706 per unit cell. In many (if not all) of the embodiments of the present invention, the cavity that is created by packing diamondoid or heterodiamondoid molecules into a crystal may be too small to accommodate a transition element metal, but crystallization around a transition element, such as gold, may occur such that the conductivity of the material is enhanced. There may be none, or more than one pore in molecular crystals of other diamondoids, and the sizes of these pores may vary.

The significance of the packing of the exemplary [1(2,3)4] pentamantanes illustrated in FIG. 7 is that biological probe may be fabricated with little further processing than the isolation techniques that use chromatography, with the exception of a functionalization step, such that the probe has active chemical groups on its surface for binding to analyte target molcules.

It is also contemplated that some polymerization reactions may be useful in creating a solid comprising various amounts of the above mentionned molecular crystals. Further information on the synthesis of diamondoid containing polymers is provided in a U.S. Patent Application titled “Polymerizable Higher Diamondoid Derivatives,” Ser. No. 10/046,486, filed Jan. 16, 2002, incorporated herein by reference in its entirety.

Nitrogen-Vacancy and Dopant Atom Color Centers in Diamond

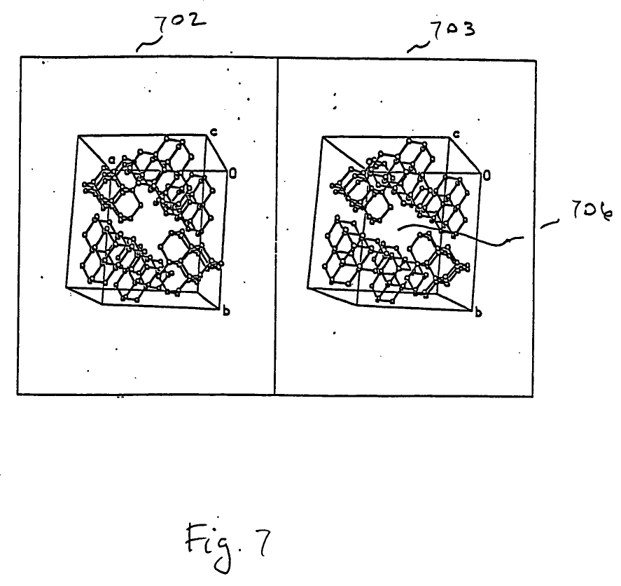

Nitrogen aggregates in diamond have been discussed by I. Kiflawi et al. in “Theory of aggregation of nitrogen in diamond,” Properties, Growth and Applications of Diamond, edited by M. H. Nazaré and A. J. Neves (Inspec, London, 2001), pp. 130-133. These authors teach that nitrogen is the major impurity in both natural and synthetic diamond. It is found both in dispersed form and aggregated form. A flowchart showing the relationship amongst the different types of diamond, based on the state of nitrogen aggregation, is given in FIG. 8. In the actual nitrogen aggregation sequence, nitrogen is incorporated into the diamond lattice as a single substitution on a diamond lattice site. As the nitrogen aggregation sequence continues, other nitrogen-containing centers are produced that are associated with greater numbers of vacancies. Such centers include the H3 center, the N3 center, and the B-center. In nature, nitrogen aggregates (and their associations with vacancies) are formed as a result of a process that takes place over geologic time scales at temperatures which prevailed within the earth's upper mantle. This view is supported by a laboratory experiments in which diamonds annealed at high temperatures displayed the same aggregates.

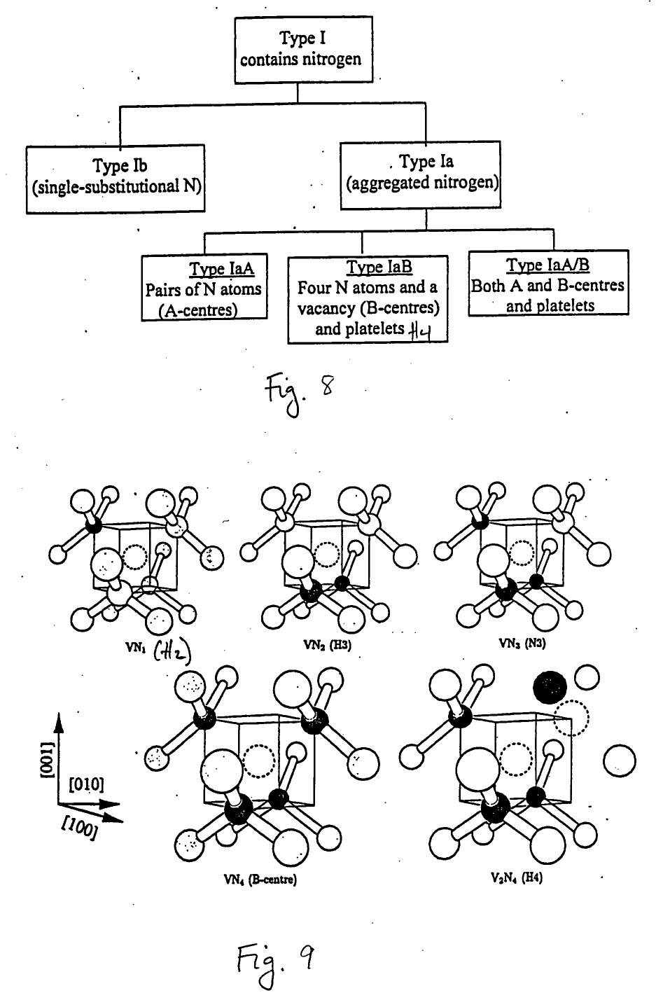

Nitrogen-vacancy associations have also been discussed by R. Jones et al. in “Theory of aggregation of nitrogen in diamond” in Properties, Growth and Applications of Diamond, edited by M. H. Nazaré and A. J. Neves (Inspec, London, 2001), pp. 127-129. This paper reviewed properties including the energies and lifetimes of optical transitions, local vibrational modes and vibrational resonances to study the structure of such color centers. Various types of aggregated nitrogen, and nitrogen vacancy complexes are illustrated in FIG. 9. An association between a single nitrogen atom and a single lattice vacancy is designated a VN₁ center, also called an H2 center. Those skilled in the art will note that the nitrogen impurity atom has substitutionally replaced one of the four carbons in a tetrahedrally coordinated around the vacancy. In the VN₂ center, also termed an H3 center, a single lattice vacancy has tetrahedrally coordinated around it two nitrogen atoms substitutionally positioned on diamond lattice sites. The VN₃ center, also known as an N3 center, consists of three nitrogen atoms tetrahedrally positioned around a single vacancy. In the VN₄ center, or B-center, all four tetrahedral positions surrounding a single vacancy are occupied with nitrogen atoms.

Color centers in diamond have been discussed by Anthony et al. in U.S. Pat. No. 6,377,340. Anthony teaches that ultraviolet light can excite color centers in diamond, causing them to luminesce or fluoresce in the visible spectrum. Luminescence from color centers in diamond can be suppressed by a high concentration of A centers. If an A center is near a color center of the diamond, the ultraviolet energy that is absorbed by the color center will not re-radiate as fluorescence or photoluminescence. Rather, the ultraviolet energy that is absorbed by the diamond's color center will be transferred to the A center, and undergo a non-radiative decay. A lattice vibration (in the form of phonons or heat) may be emitted from the diamond rather than visible light when an A center is positioned adjacent to an excited color center that has absorbed ultraviolet light. Typical color centers in diamond that may be excited by ultraviolet light include the N3 centers and the H3 centers.

The light emitting properties of diamond have been discussed by Satoh et al. in U.S. Pat. No. 4,880,613. Pure diamond containing no impurities does not absorb or emit light even in the ultraviolet wavelengths. Therefore, color centers have to be created in the diamond crystal. To create such color centers, the nitrogen atoms contained in the diamond are converted to one or more of the following four types:
1) Ib type (discrete dispersion type)
2) IaA (two nitrogen atoms aggregate)
3) IaB (four nitrogen atoms aggregate).

Alternatively, the nitrogen and impurity atoms may be combined with a lattice site vacancy to create the following types of color centers:
4) N-V color center (Ib type nitrogen-vacancy)
5) H3 color center (IaA type nitrogen-vacancy)
6) H4 color center (IaB type nitrogen-vacancy).

The wavelengths of the emitted light from these types of color centers are 638-780 nm, 503-600 nm, and 494-580 mm, respectively.

Satoh et al. in U.S. Pat. No. 4,880,613 contain to disclose that an N-V center (nitrogen-vacancy center) may be formed by combining a type Ib type nitrogen atom with a lattice site vacancy. To form an N-V center in diamond, the material is irradiated by electron beam or a neutron beam to generate lattice vacancies. Then, the irradiated diamond is annealed by heating in vacuum to position the lattice vacancy adjacent to the nitrogen atom to form the N-V center.

In an article entitled “Stable solid-state source of single photons,” by C. Kurtsiefer et al., Physical Review Letters, Vol. 85, No. 2, pp. 290-293 (Jul. 10, 2000), fluorescence light observed from a single nitrogen-vacancy center in diamond is discussed. Such a center exhibits strong photon antibunching and only one photon is emitted at a time. Nitrogen-vacancy centers are reported to be well localized, and stable against photobleaching even at room temperature.

Kurtsiefer et al. report that N-V centers are one of the many well studied luminescent defects in diamond and that they may be formed by substitutionally positioning a nitrogen atom with a vacancy trapped at an adjacent lattice position. Usually, the centers are prepared in type Ib synthetic diamond, where single substitutional nitrogen impurities are homogeneously dispersed. To obtain bright luminescence from a sample, additional vacancies are created by electron or neutron radiation. The vacancies are then allowed to diffuse to the nitrogen atoms by annealing at 900° C. These authors report, however, that untreated samples of synthetic type Ib diamond provides a concentration of N-V centers that are well suited for addressing the properties of individual color centers. The high radiative quantum efficiency, even at room temperature, at close to one, coupled with a short decay time of the excited state, make them a well-suited for single photon generation.

A photoemissive device employing a diamond having H3 and N3 color centers as a lasing medium has been discussed by Rand et al. in U.S. Pat. No. 4,638,484. Rand disclosed the demonstration of laser action in natural Type I diamonds containing H3 and N3 color centers when excited by an optical pumping source comprising a light source emitting ultraviolet radiation in the 300-600 nm range. High concentrations of N3 color centers emitted a bright blue fluorescence, while high concentrations of H3 centers emitted a bright green-yellow fluorescence. The diamonds suitable for use as laser active materials contained nitrogen substitutions at a level of at least 0.1 atomic percent. The gain coefficient of the H3 centers was calculated as 0.09 cm⁻¹, while the gain coefficient for the N3 centers was estimated at about 0.009 cm⁻¹.

Another photoemissive device comprising H2 centers has been described by Satoh et al. in U.S. Pat. No. 4,949,347. Laser action was effected in the range 1000 to 1400 nm by an external light pumping source operating at 650 to 950 nm. One method for providing the lasing medium material comprised the steps of subjecting a synthetic type Ib diamond having a nitrogen concentration within the range of 1×10¹⁷ to 8.5×10¹⁹ atoms/cm³, irradiating the nitrogen-containing diamond with an electron dose of not less than 5×1017 electrons/cm², followed by a heat treating step. The heat treating method was optionally performed under ultra high-pressure of not less than 3.0 GPa, and high-temperature conditions of not less than 1500° C. The diamond laser was activated using a semiconductor laser(s) as the source of external pumping. For laser action using H2 centers, it was necessary to maintain the maximum value of the optical density of the H2 centers between 0.01 and 4, where optical density is defined as the natural log of the ratio of the incident light intensity to the transmitted light intensity. When the pumping wavelength of Satoh et al.'s diamond laser was varied between 500-1000 nm, laser action was observed in the range 1000 to 1400 nm.

A method of preparing a diamond laser crystal with a large quantity of H3 centers in synthetic Type Ib (single substitutioal N) diamond has been disclosed by Nakashima et al. in U.S. Pat. No. 4,950,625. This method involved first preparing synthetic Type 1b containing at least 60 percent of a (111) growth plane, and then thermally treating that material under high temperature/high pressure conditions such that the type Ib diamond was converted to type IaA (pairs of N atoms; see FIG. 8). The type IaA diamond was then exposed to an electron beam in order to generate vacancies. Finally, an annealing step was performed to form H3 centers by coupling the type IaA nitrogen atoms with the vacancies. The number of VN₁ centers was low, which was found to be desirable, as these are normally an obstacle to laser action.

These methods of producing color centers in diamond may be cumbersome and expensive to implement, and it may difficult to control the type, number, and distribution of color centers within the material. What is needed is an improved type of color centers in diamond materials, and methods of manufacturing the same, wherein control over the type, number, quality, uniformity, and distribution of the color centers is readily achievable.

Luminescence in Heterodiamondoid-Containing Materials

In one embodiment of the present intention, a nitrogen-containing heterodiamondoid is capable of photoluminescence by virtue of the fact that the nitrogen atom is positioned on the surface of the molecule, where surface states enable nitrogen to photoluminescence.

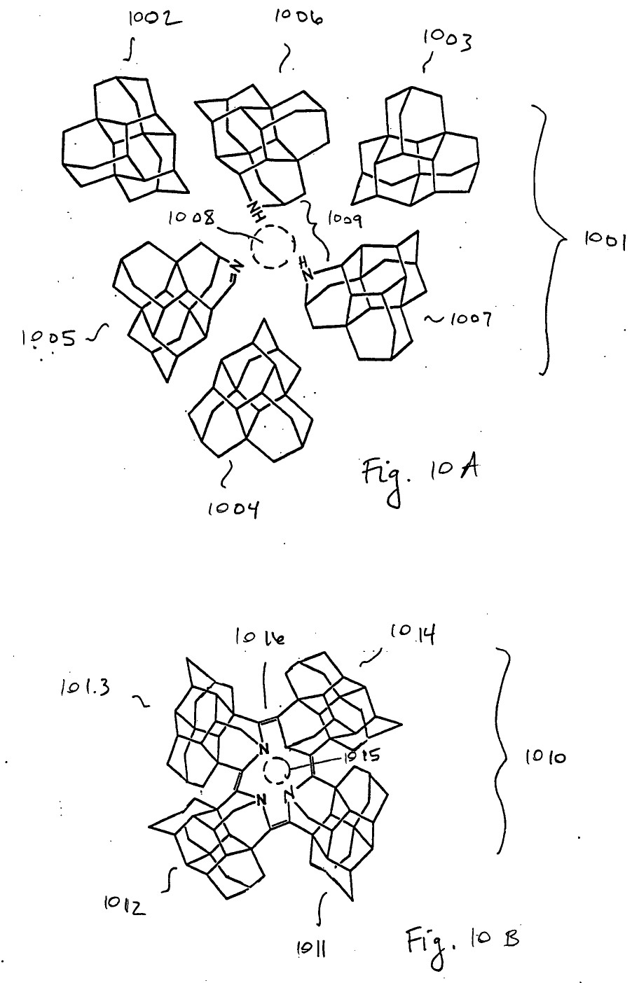

According to another embodiment of the present invention, a photoluminescing medium may be fabricated by allowing diamondoids, nitrogen-containing heteroatom diamondoids, derivatized diamondoids, and derivatized heterodiamondoids to crystallize into a molecular solid. It is contemplated that the nitrogen heteroatoms may be positioned in the solid adjacent to pores and or vacancies such that a nitrogen-vacancy (or nitrogen-pore) association is formed, wherein the number of nitrogens and the number of vacancies (or pores) in the color center assembly may be engineered according to the particular structure desired. This of course determines the properties of the light emitted. In one embodiment of the present invention, an H3 or N3 structure is approximated. Such a photoluminescent color center is contemplated in FIGS. 10A and 10B, where a molecular crystal held together substantially by van der Waals forces is depicted in FIG. 10A, and a covalently bonded diamondoid polymer is depicted in FIG. 10B.

Referring to FIG. 10A, a diamondoid-containing material suitable for use as a biological label having a nitrogen-vacancy or nitrogen-pore color center is depicted generally at 1001. Individual diamondoids 1002, 1003, and 1004 pack with individual heterodiamondoids 1005, 1006, and 1007 forming a pore 1008 generally at the center of the group. Heterodiamondoids 1005, 1006, and 1007 pack, assemble, or are otherwise constructed such that their nitrogen heteroatoms are generally positioned adjacent to pore or vacancy 1008 forming a structure that resembles an N3 color center 1009. It will be understood by those skilled in the art that many possible combinations of pore sizes, types of heteroatom bonding within each heterodiamondoid, valence structure of each heteroatom within the heterodiamondoid, geometrical positioning and configuration of diamondoids and heterodiamondoids to one another, packing density of diamondoids, etc., are possible. Thus, it is possible to control the optical properties of the color center 1009 within the molecular crystal 1001 to achieve the desired photoluminescing light properties.

Referring to FIG. 10B, a diamondoid-containing material shown generally at 1010 comprises heterodiamondoids 1011, 1012, and 1013, and diamondoid 1014. Heterodiamondoids 1011, 1012, and 1013, contained nitrogen heteroatoms. These four diamondoids may be held in a covalently bonded structure according to the techniques described for the polymer in FIG. 6. The polymerization synthesis is carried out such that the nitrogen heteroatoms of the heterodiamondoids 1011, 1012, and 1013, respectively, are positioned adjacent to a pore, opening, or vacancy 1015. The nitrogen heteroatoms and pore 1015 form a color center 1016 located substantially at the center (in this example) of the covalently bonded structure. The pore does not have to be at the center of the structure. It will be understood by those skilled in the art that many combinations of covalent bonding structure, choice of heterodiamondoids, degree of sp² vs. sp³ character in the covalent bonding, etc., are possible. Thus, it is possible to control the optical properties in the color center 1016 within the polymerized material 1010.

Advantages of the present molecular crystals and polymerized diamondoids are that the nitrogen-vacancy-containing color center are constructed “from the bottom up,” meaning that the nitrogen heteroatoms and the vacancies comprising the color centers are placed in position by virtue of the details of the assembling technique, whether crystallization or polymerization. This may be contrasted with the damaging techniques of the prior art methods, wherein nitrogen atoms are either already present in the crystal, and there is less control over their density or distribution, or inserted by lattice-damaging implantation techniques. Vacancy insertion by ion beam exposure is also more likely to damage a crystal than the synthesis and assembly techniques of the present embodiments. However, it may still be possible to create a lattice site vacancy in a diamondoid and/or heterodiamondoid using either electron beam or neutron beam radiation.

The present embodiments include a biological label which may comprise a diamondoid-containing probe, a light source for delivering energy to the biological label, and a detection system for processing the light emitted from the biological label. The biological probe may comprise a diamondoid or diamondoid-containing material having at least one color. The color center may comprise at least one nitrogen-containing heteroatom in a heterodiamondoid, where the heteroatom may be positioned adjacent to at least one vacancy or pore.

The diamondoid-containing materials contemplated by the present embodiments may comprise an individual diamondoid, and individual heterodiamondoid, a molecular crystal, a polymerized material, and various combinations thereof. The diamondoid may be selected from the group consisting of adamantane, diamantane, and triamantane, and heterodiamondoid derivatives thereof. It may also comprise at least one higher diamondoid selected from the group consisting of tetramantane, pentamantane, hexamantane, heptamantane, octamantane, nonamantane, decamantane, and undecamantane, and heterodiamondoid derivatives thereof.

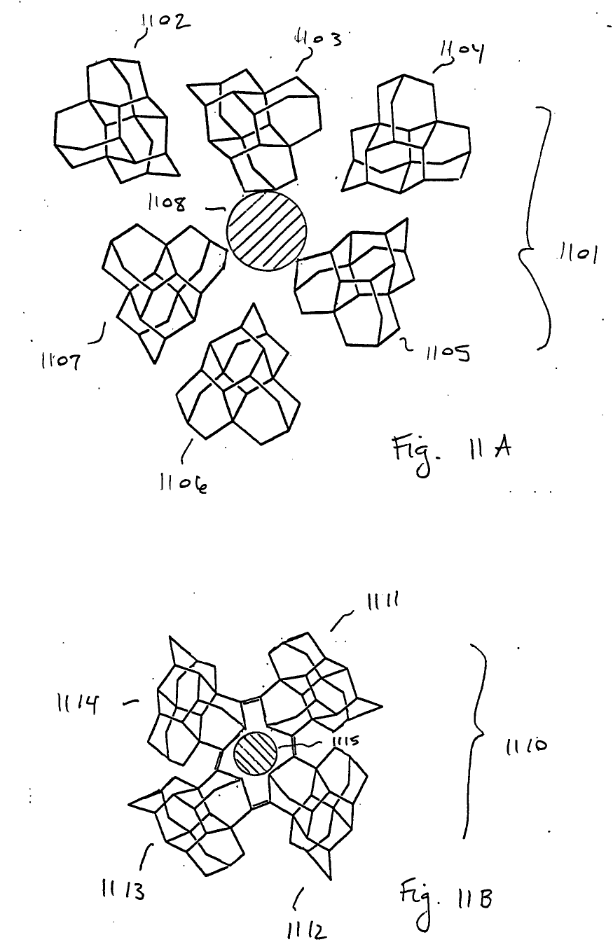

In an alternative embodiment, a diamondoid-containing molecular crystal or polymeric biological probe may include a dopant impurity for photoluminescence. The dopant may be a rare earth element, transition element, actinide, or lanthanide. Photoluminescent dopants may be inserted into a diamondoid-containing material according to present embodiments by self-assembly, crystallization, and polymerization techniques similar to those used for nitrogen-vacancy color centers. An exemplary self-assembled or crystallized material suitable for use in a biological label is shown generally at 1100 in FIG. 11A. Diamondoids 1102-1107 may be generally disposed around an optically active dopant 1108. The photoluminescent dopant 1108 may comprise a rare earth element, transition element, actinide, or lanthanide, or mixtures thereof. The optically active dopant may be selected from the group consisting of titanium, vanadium, chromium, iron, cobalt, nickel, zinc, zirconium, niobium, cadmium, hafnium, tantalum, tungsten, rhenium, osmium, iridium, platinum, gold, mercury, cerium, praseodymium, neodymium, promethium, samarium, europium, gadolinium, terbium, dysprosium, holmium, erbium, thulium, ytterbium, and uranium. Some of the diamondoids surrounding the optically active dopant 1108, and comprising the pocket in which the dopant sits, may be either positioned in close proximity to the dopant atom, in contact with it, or even bonded to it in some manner, such as through a covalent or ionic bond, or through London forces. Exemplary diamondoids in FIG. 11A include 1103, 1105, and 1107. Other diamondoids comprising the pocket may be positioned further away from the dopant atom; such diamondoids include 1102, 1104, and 1106. These more distant diamondoids may also exert a force on the dopant, or no force at all. The dopant atom may also be chemically inert with respect to its diamondoid hosts. Of course, in keeping with the definition of diamondoids in this disclosure, the diamondoids may also be heterodiamondoids, or derivatives thereof.

A polymerized diamondoid-containing material that may host an optically active dopant atom is shown generally at 1110 in FIG. 11B. This exemplary material comprises four diamondoids 1111-1114 that form a pore within which an optically active dopant atom 1115 resides. As with the molecular crystal 1101, any of the diamondoids 1111-1114 that comprise polymerized material 1110 may contact or be bonded in some manner to the dopant atom, or they may be chemically inert to it and the optically active dopant atom 1115 may be held in place mechanically.

Control of the frequency of the emitted light and quantum efficiency Traditional methods for detecting biological compounds in vivo and in vitro have been disclosed by Bawendi et al. in U.S. Pat. No. 6,306,610, and by Bawendi et all in U.S. Pat. No. 6,326,144. Some of these methods have involved the use of organic fluorescent dyes, which have chemical and physical limitations. For example, one limitation is the variation of excitation wavelengths of different color dyes. As a result, simultaneously using two or more florescent tags with different excitation wavelengths requires multiple excitation sources. Another drawback with the use of organic dyes is the deterioration of fluorescence intensity upon prolonged exposure to the excitation light source. This fading is called photobleaching and is dependent on the intensity of the excitation light and the duration of the illumination. In addition, conversion of the dye into a nonfluorescent species is reversible. Furthermore, the degradation products of organic dyes are organic compounds which may interfere with the biological processes being examined.

Bawendi et al. disclose that a further drawback of organic dyes is that there can be a spectral overlap from one dye to another. This is due in part to the relatively wide emission spectra of organic dyes and the overlap of the spectra near what is known as the tailing region. The ideal florescent label should fulfill requirements such as high florescent intensity, a separation of at least 50 nm between the absorption and florescent frequencies, solubility in water, the ability to be linked readily to other molecules, a stability toward harsh conditions and high temperatures, and a symmetric and gaussian peak shape for easy deconvolution of multiple photoemitted frequencies.

Quantum dots are know in the art, and have been defined by Bawendi et al. in U.S. Pat. No. 6,326,144 as semiconductor nanocrystals with size dependent optical and electronic properties. A particularly important property of quantum dots is that their bandgap energy can vary with the size of the crystal. The semiconductor nanocrystal has a characteristic spectral emission, which is tunable to a desired energy by selection of the particle size of the quantum.

Another description of quantum dots has been given by Bawendi et al. in U.S. Pat. No. 6,322,901. Bawendi et al. teach that semiconductor nanocrystallites have radii smaller than the bulk exciton Bohr radius to cause quantum confinement of both electrons and holes in a three-dimensional manner within the material; this leads to an increase in the effective bandgap of the material without requiring a decrease in crystallite size. Both the optical absorption and emission spectra of such quantum dots are shifted toward higher energies as the size of the crystallites gets smaller. The photoluminescent yield of such crystallites can be poor (that is to say, the intensity of the light emitted upon radiation is low) because of energy levels at the surface of the crystallite that lie within the energetically forbidden bandgap of the bulk interior. These surface energy states act as traps for electrons and holes which degrade the luminescence properties of material.

Bawendi et al. further teach that photoluminescent yield of the quantum dots may be improved by passivating the surface with organic ligands to eliminate forbidden energy levels that lie within the bandgap. Passivation of quantum dots using inorganic materials has also been reported. This patent teaches the preparation of highly luminescent ZnS-capped CdSe nanocrystallites having a narrow particle size distribution.

The size of the semiconductor core, and its correlation with the spectral range of emissions, has been been reported in U.S. Pat. No. 6,309,701 to Barbara-Guillem. This data reports the peak emission range of a Group II-VI semiconductor core; e.g., ZnS or CdSe, passivated with a shell comprised of YZ, wherein Y is Cd or Zn, and Z is S or Se. For example, a core having a size range of 2.5 to 2.68 nm emits blue colored light in the range of 476 to 486 nm, and a core having a size range of 8.6 to 10.2 emits red colored light in the range 644 to 654 nm.

The functionalized heterodiamondoid probes contemplated by the present embodiments have their emission frequencies adjustable by the selection of a particular diamondoid. Alternatively, the size of the probe may be adjusted by the number of heterodiamondoids crystallized into a particular molecular solid, and or by the number of heterodiamondoids polymerized into a particular oligomeric solid. It is contemplated that by varying molecular crystal size; i.e., the extent of the molecular aggregation, degree of crystal growth, and/or choice of diamondoid(s), the desired fluorescent spectral distribution may be acquired. Furthermore, the use of impurities that contribute electronic states within the band gap will allow for the adjustment of the frequency of the emitted light. It is believed that the bandgap(s) of the present materials is at least about 5 eV, approaching the value for bulk diamond, and thus a wide frequency spectrum is believed to be available, ranging from the infrared, through the visible, to the ultraviolet. However, the bandgap of the present materials may also be engineered to be, in respective embodiments, at least about 2 eV, 3 eV, 4 eV. It is contemplated that the band gap of higher diamondoids may show a quantum confinement effect similar to that of a quantum dot.

Furthermore, it is contemplated that the quantum efficiency of the heterodiamondoid probe may be influenced by passivating the surface of the functionalized heterodiamondoid, molecular crystal comprising functionalized heterodiamondoids, or polymerized solid comprising functionalized heterodiamondoids, with the appropriate choice of passivating agents. Additionally, such passivation may enhance water solubility of the probe.

Biological Labels

Embodiments of the present invention include a biological probe that can provide information about a biological state or event. The probe can detect the presence or amounts of a biological moiety; the structure, composition, and conformation of the biological moiety; the localization of the biological moiety in an environment; interactions of biological moieties; alterations in structures of biological compounds; and alterations in biological processes.

The probe comprises a functionalized heterodiamondoid capable of exhibiting a photoluminescence event, wherein the functionalized heterodiamondoid has an affinity for a biological target. The probe interacts or associates with the biological target due to the affinity of the compound with the target. At this stage, the target has been “labeled.” The location and the nature of the labeled target can be detected by monitoring the emission of light from the functionalized heterodiamondoid while it is in the state of being bound to or associated with the target.

In operation, the probe is introduced into an environment containing the biological target and the probe associates with the target. The probe/target complex may be spectroscopically viewed by radiation of the complex with an excitation light source. The labeled target emits a characteristic spectrum which can be observed and measured.

It is contemplated by the present invention that a plurality of functionalized heterodiamondoids as part of a larger system may be simultaneously excited with a single light source, usually in the ultraviolet or blue region of the spectrum. The functionalized heterodiamondoid biological probes of the present invention are contemplated to be more robust than conventional organic fluorescent dyes of the prior art, and more resistant to photobleaching than such dyes. Furthermore, the robustness of the probes of the present invention will likely alleviate the problem of contamination caused by the degradation products of the organic dyes being used. Therefore, biological labels based on functionalized heterodiamondoids are expected to provide a unique source of valuable tags for the detection of biological molecules, and the interactions they undergo.

According to embodiments of the present invention, the functional groups of the heterodiamondoid probe allow the heterodiamondoid to physically interact with the biological molecules of interest (i.e., the targets). Without limiting the scope of the invention, the functional groups of the heterodiamondoids can bind to proteins, nucleic acids, cells, subcellular organelles, lipids, carbohydrates, antigens, antibodies, nucleic acids, and other biological molecules. The affinity between the functional groups of the heterodiamondoid probe and the target molecule (hereinafter referred to as target analyte or simply analyte) may be based upon any of a different number of binding schemes or associations, including but not limited to van der Waals attractions, hydrophilic attractions, hydrophobic attractions, ionic and/or covalent bonding, and electrostatic, and/or magnetic associations. As used herein, “biological target” or “target analyte” is means any chemical moiety of biologic origin, compound, cellular or subcellular component which is associated with a biological function. The biological target includes without limitation proteins, antigens, antibodies, nucleic acids, cells, subcellular organelles, and other biological moieties.

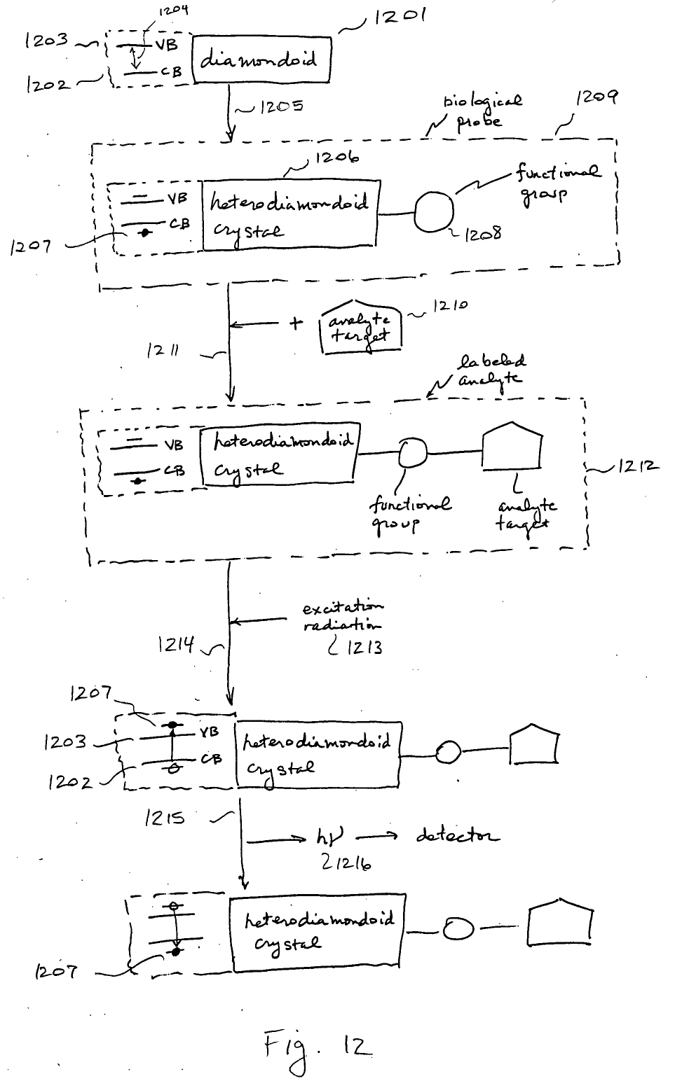

The operation of the probe is illustrated in FIG. 12. Referring to FIG. 12, a diamondoid 1201 is shown relative to an energy scale (with increasing energy pointing upwards), an empty conduction band 1202 (CB), and an empty valence band 1203 (VB). It will be understood by one skilled and art that there are of course occupied electronic states in the conduction band, but since the carbon atoms position on diamond lattice sites utilize each other for valence electrons for tetrahedral bonding, there are a few excess of electrons available for excitation across the bandgap 1204 to the valence band 1203 at room temperature.

In a processing step 1205, the diamondoid 1201 is converted to a heterodiamondoid 1206, or in at least one carbon, diamond lattice site is replaced by nitrogen. Since nitrogen lies one column to the right of carbon in the periodic table, it has excess electron relative to carbon. This is shown schematically by the electron 1207 in the heterodiamondoid 1206. As described above, the heterodiamondoid 1206 may be derivatized with at least one functional group 1208. The functionalized heterodiamondoid entity constitutes a biological probe 1209.

The probe 1209 may be reacted with an analyte target 1210 in a process step 1211 to form a probe/target complex 1212. Consistent with the nomenclature used herein, the analyte 1210 is now labeled because it is associated with the functionalized heterodiamondoid (probe) 1209.

To detect the presence of analyte 1210, the labeled analyte 1212 is exposed to excitation radiation 1213 and a step 1214. This has the result of exciting the electron 1207 across the bandgap 1204 from the conduction and 1202 to the valence band 1203. In a subsequent step 1215a photon 1216 is emitted from the probe/target complex as a result of the photoluminescent decay of electron 1207 back to the conduction and 1202. Note that the energy states in a valence band and convection band have been depicted only very loosely in terms of the energy levels, and should not be strictly interpreted in the schematic FIG. 12. In other words, the energy diagrams in FIG. 12 are not meant to indicate that the amount of energy absorbed in 1214 is the same as the amount of energy emitted in 1215; rather, FIG. 12 is merely meant to convey the fact that energy is either being absorbed and then emitted by the system.

Conjugation of the Heterodiamondoid to a Target

As discussed by G. T. Hermanson in “Bioconjugate Techniques” (Academic Press, San Diego, 1996), in the preface to the book, bioconjugation involves the linking of two or more molecules to form a novel complex having the combined properties of the individual components. It is contemplated that the heterodiamondoids of the present embodiments may be linked to the target analytes such as proteins, polysaccharides, nucleic acids, lipids, and virtually any other imaginable molecule that can be chemically functionalized.

The binding of the present heterodiamondoid-containing biolabels to proteins may be effected by techniques discussed in Chapter 1 of Bioconjugate Techniques. In this chapter it is disclosed that proteins may contain up to nine amino acids that are readily derivatizable at their side chains, and that the nine residues contain eight principal functional groups with sufficient reactivity for modification rections: primary amines, carboxylates, sulfhydryls (or disulfides), thioethers, imidazolyls, guanidinyl groups, and phenolic and indolyl rings.

For example, it is disclosed by G. T. Hermanson that carboxylate groups in proteins may be derivatized through the use of amide bond forming agents or through active ester or reactive carbonyl intermediates. The carboxylate becomes the acylating agent to the modifying group. It is further disclosed that amine containing nucleophiles can couple to an activated carboxylate to give amide derivatives. As discussed in U.S. patent application Ser. Nos. 10/313,804, and 10/046,486 (incorporated herein by reference in their entirety), the functionalized higher diamondoids may be derivatized with any of the moieties —H, —F, —Cl, —Br, —I, —OH, —SH, —NH₂, —NHCOCH₃, —NHCHO, —CO₂H, —CO₂R′, —COCl, —CHO, —CH₂OH, ═O, —NO₂, —CH═CH₂, —C≡CH and —C₆H₅; where R′ is an alkyl group, preferably ethyl.

These functional groups on the diamondoid provide the chemistry that may be used for binding to the protein. The diamondoid fuctional groups may react with either the side chain functional groups of the amino acids, or they may react with either the N-terminal α-amino and the C-terminal α-carboxylate groups. which provides the chemistry that may be used for binding to the protein.

The principle sites of reactivity on carbohydrates for conjugation purposes is also discussed in Bioconjugate Techniques. For example, monosaccharide functional groups consist of either a ketone or an aldehyde, several hydroxyls, and the possibility of amine, carboxylate, sulfate, or phosphate groups as additional reactive possibilities. Sugar hydroxyl groups may be derivatized by acylating or alkylating reagents, similar to the reactions of primary amines. Other exemplary reactions that may be used to bind to the functionalized heterodiamondoids include oxidizing hydroxyl groups to form reactive formyl groups; conjugating the native reducing ends of carbohydrates to amine-containing diamondoids by reductive amination; modifying the reducing ends of oligosaccharides to yield terminal arylamine derivatives; forming hydrazone linkages; creating aldehyde functional groups, and subsequently derivatizing them with another molecule containing an amine or a hydrazide. The hydroxyl residues of polysaccharides may be activated to form good leaving groups for nucleophilic substitution.

Similarly, nucleic acids may be conjugated to a functionalized heterodiamondoid(s) to generate the biolabels of the present embodiments. Nucleic acids can contain any one of three types of pyrimidine ring systems (uracil, cytosine, or thymine), and two types of purine derivatives (adenine or guanine); along with nucleic acid sugar residues which are attached to the asswociated base units in an N-glycosidic bond. The sugar group consists of either a β-D-ribose unit (found in RNA) or a β-D-2-deoxyribose unit (found in DNA). In each nucleotide monomer of DNA or RNA, a phosphate group is attached to the C-5 hydroxyl of each sugar residue in an ester (anhydride) linkage. The phosphate groups are then in tern linked in diester bonds to neighboring sugar groups of adjacent nucleotides through their 3′-ribosyl hydroxyl to create the oligonucleotide polymer backbone.

As further pointed out by G. T. Hermanson, chemical attachment of a detectable component to an oligonucleotide forms the basis for constructing a sensitive hybridization reagent. There are particular sites on the bases, sugars, or phosphate groups of nucleic acids that can be derivatized to react with the functional groups of the heterodiamondoid. For example, cytosine, thymine, and uracil all react toward nucleophilic attack at the C-4 and C-6 positions. Adenine and guanine residues are susceptible to nucleophilic displacement reactions at the C-2, C-6, and C-8 positions, with C-8 being the most common target for modification. Conjugation may be done on the sugar groups through the 3′hydroxyl group of the deoxyribonucleic acids, or the 2′,3′-diol of the ribonucleid acids. Two possible conjugation reactions that are possible at the phosphate include condensation agents such as carbodiimides, and conversion of the phosphate group to a phosphoramidite derivative.

Conjugation of the present heterodiamondoids is not limited to proteins, carbohydrates, and nucleic acids, and many other types of target molecules are contemplated. These include, but are not limited to, subcellular organelles, lipids, antigens, antibodies, dyes, and other biological molecules

Bioavailability and Membrane Transport

It is contemplated that the biological labels of the present invention may be used in applications where it is desired to assay a target analyte in an intra-cellular or in-vitro situation. In such an application, the present biolabels need the ability to be transported either actively or passively across the cell membrane. It should be emphasized that cell membrane permeation by the biolabel is only one embodiment contemplated by the present invention, and may extra-cellular and in-vitro applications for the present biolabels may also be envisioned.

The cell transport properties of adamantine (1-amino adamantane, C₁₀H₁₇N) have been discussed by Roger K. Murray, who has stated that “amantadine enters all cell membranes, crosses the blood-brain barrier, and has nearly ideal pharmacokinetic and metabolic profiles.” A further discussion of membrane permeation has been provided by Verber et. al. (GlaxoSmithKline), who has disclosed that membrane permeation is recognized as a common requirement for oral bioavailability in the absence of active transport, and failure to achieve this usually results in poor oral bioavailability. Verber's work included making measurements of the oral bioavailability in rats of over 1,100 drug candidates. The results showed that key molecular properties such as reduced molecular flexibility, as measured by the number of rotatable bonds, low polar surface area or total hydrogen bond count, are found to be good predictors of oral bioavailability.

This finding is in contrast to the generally held belief that size, or molecular weight, is a critical factor in determining bioavailability. On average both the number of rotatable bonds and the amount of surface area of the biolabel that is polar (or hydrogen bond count) tend to increase with increasing molecular weight, and this may in part explain the success of molecular weight as a parameter in predicting oral bioavailability. The commonly applied molecular weight cutoff of 500 does not itself significantly separate compounds with poor bioavailability versus those with good bioavailability.

The biolabels of the present embodiments are contemplated to possess desirable properties relating to bioavailability, in part because of the manner in which a molecule's physical predicts bioavailability. As defined by Verber et al., these properties may include the number of rotatable bonds the biolabel possesses, the number of hydrogen bond donors or acceptors, and the amount of polar surface area of the label.

Verber defines rotatable bonds to be any single bond, not in a ring, bound to a nonterminal heavy (i.e. non-hydrogen atom), and the heterodiamondoid-containing materials of the present embodiments may contain virtually no rotatable bonds. It is noted that C—N bonds were excluded from Verber's analysis because of their high rotational energy barrier. Hydrogen bond donors were defined to be any heteroatom with at least one bonded hydrogen, whereas hydrogen bond acceptors were defined to be any heteroatom without a formal positive charge, excluding halogens, pyrrole nitrogen, heteroaromatic oxygen and sulfur, and higher oxidation states of nitrogen, phosphorous, and sulfur but including the oxygens bonded to them.

Polar Surface Area may be calculated by the atom-based method of Ertl, Rohde, and Selzer, in an article entitled “Fast calculation of molecular polar surface area is done as a sum of fragment-based contributions and its application to the prediction of drug transport properties,” J. Med. Chem. 2000, vol. 43, pp. 3,714-3,717. The calculated polar surface area correlated closely with the total hydrogen bond count, the sum of hydrogen bond donors and acceptors. For the oral bioavailability data set, r was found to be equivalent to 0.93.

It is contemplated that the biolabels of the present embodiments will have advantageous bioavailability properties because they meet Verber's requirements of about 10 or fewer rotatable bonds, and less than about 140 square angstroms of polar surface area, or alternatively, 12 or fewer H-bond donors and acceptors. This is particularly true for the biolabel shown in FIG. 10B, the fluorescing portion of that biolabel comprising a cluster of four tetramantanes with at least one nitrogen-based heteroatom for desired optical properties. Of course, it will be recognized by those skilled in the art that diamondoids other than tetramantane may also be used. The advantages of the present biolabels include the extraordinary rigidity of the diamondoid portion of the label, and the relative lack of flexible structures such as rotatable bonds.

In one embodiment of the present invention, the biolabel comprises at least four diamondoid structures of tetramantane or higher, having fewer than about 25 rotatable bonds, less than about 500 total polar surface area, square angstroms of polar surface area, or alternatively, 25 or fewer H-bond donors and acceptors. A molecular weight estimate of about 1,200 for a biolabel comprising four tetramantanes (C₂₂H₂₈, each having a molecular weight of 292) and at least one nitrogen heteroatom to provide a fluorescing color center) is contemplated to be within the weight limits (according to Verber's calculations) for molecules having good bioavailability.

Optical Detection Systems

According to some embodiments of the present invention, light emitted from the biolabel is detected using photechniques known in the art. The fundamental steps in the contemplated fluorescence-based detection system are:
1. Excitation light delivery or to excite the fluorescent dyes on the sample;
2. Emission light collection or to collect the emitted light; and
3. Digital image generation of the fluorescent signal.

Two general methods may be used in the present embodiments to acquire such images: laser excitation in conjunction with a photomultiplier tube (PMT) detector, and filtered white-light excitation with a charge-coupled device (CCD) detector. In addition, the laser-based systems can use either a confocal or nonconfocal optical path. In this section of the disclosure the excitation light delivery systems will be discussed first, followed by the emission light collection systems, and digital image generation techniques. The section will conclude with a discussion of confocal versus nonconfocal optics, and their relevance to the present biolabels.

Turning first to a discussion of excitation light delivery systems, a laser-based system may be used wherein a single-wavelength laser beam of a few microns in diameter is scanned back and forth across a sample, exciting an area representing a single pixel at a time. Emission light travels back through the excitation lens and is collected by the PMT. The PMT amplifies the signal from each photon, which is then converted into a digital value used to create an image representing the signal intensity at each pixel position.

In a white-light system, a broad-spectrum white-light source such as a xenon or mercury lamp provides the excitation light. The excitation wavelength is selected by filtering the white light into a narrower wavelength range. The lamp illuminates a large area of the sample, and the fluorescent emission from the entire field of view is collected by a stationary CCD array. An imaging aperture is opened for varying times to allow the CCD to collect enough light from the sample to create a representative image. The signal intensity at each pixel position on the CCD array is then converted into a digital image.

Laser illumination concentrates high-power monochromatic light in a small spot at the sample surface. The higher power density delivers more light to the fluorescent molecule, therefore much less time is required to excite the dye than with filtered white light. As the laser beam scans the sample, it “dwells” on each pixel position for several microseconds. In contrast, a white-light source illuminates the sample for seconds or minutes while the CCD integrates the emission signal during the entire exposure time.

Turning now to techniques for the collection of the emission light: two important detector characteristics that contribute to overall system performance are linear range and quantum efficiency. Linear range indicates the range of input signal intensities over which the detector can accurately measure change, such that a given degree of change in input signal generates the same degree of change in output signal. PMTs have an optimum working linear range over which the signal response is most accurate. The linear range of a CCD detector is specified as the ratio of the capacity of each well on the CCD array to the readout noise level (i.e., random error due to fluctuations in each pixel measurement). The signal intensity range of a CCD is adjusted by changing the exposure time. Similar to a PMT, a CCD array is also linear with increasing integration time. However, dark current, or signal generated by random electrons flowing through the device in the absence of light, increases proportionally with exposure and may increase the background signal.

An important characteristic of a detector with regard to digital image generation is quantum efficiency (QE), which is a measure of the electronic signal the device emits relative to the incoming photon signal it receives. As a stand-alone component, most CCDs used in microarray imaging systems have about twofold greater QE than standard PMTs. CCD imaging systems generally capture multiple images of the sample, which are then stitched together to create a single image. Imprecise stitching, photobleaching due to multiple exposures of the overlapping regions, and other artifacts can interfere with accurate quantitation. An alternative to excessive stitching might be to use a camera-type lens to reduce a relatively large area of the microarray onto a smaller CCD surface. However, in all optical systems, if the detector is smaller than the source, losses in light collection efficiency are inevitable.

Laser-based systems can use either a confocal or nonconfocal optical pathway design. Confocal optics were originally developed to image thin sections of a thick sample, such as cells or tissue. Confocal optics create a very narrow depth of focus to reject signal from beyond that narrow focal plane. Repeated scanning at different depths creates multiple high-quality optical sections that can be reconstructed into a 3-D image of the thick sample.

All of the publications, patents and patent applications cited in this application are herein incorporated by reference in their entirety to the same extent as if the disclosure of each individual publication, patent application or patent was specifically and individually indicated to be incorporated by reference in its entirety.

Many modifications of the exemplary embodiments of the invention disclosed above will readily occur to those skilled in the art. Accordingly, the invention is to be construed as including all structure and methods that fall within the scope of the appended claims.

1. A biological label comprising at least one luminescent color center, the color center comprising a nitrogen heteroatom substitutionally positioned on a diamondoid lattice site adjacent to at least one vacancy or pore.

2. The biological label of claim 1, wherein the diamondoid is a lower diamondoid selected from the group consisting of adamantane, diamantane, and triamantane, and heterodiamondoid derivatives thereof.

3. The biological label of claim 1, wherein the diamondoid is a higher diamondoid selected from the group consisting of tetramantane, pentamantane, hexamantane, heptamantane, octamantane, nonamantane, decamantane, and undecamantane, and heterodiamondoid derivatives thereof.

4. The biological label of claim 1, wherein the diamondoid-containing material containing the nitrogen heteroatom and vacancy or pore is selected from the group consisting of a molecular crystal, a polymerized material, and combinations thereof.

5. A biological label comprising at least one optically active dopant inserted into a diamondoid-containing material.

6. The biological label of claim 5, wherein the diamondoid is a lower diamondoid selected from the group consisting of adamantane, diamantane, and triamantane, and heterodiamondoid derivatives thereof.

7. The biological label of claim 5, wherein the diamondoid is a higher diamondoid selected from the group consisting of tetramantane, pentamantane, hexamantane, heptamantane, octamantane, nonamantane, decamantane, and undecamantane, and heterodiamondoid derivatives thereof.

8. The biological label of claim 5, wherein the diamondoid-containing material containing the nitrogen heteroatom and vacancy or pore is selected from the group consisting of a molecular crystal, a polymerized material, and combinations thereof.

9. The biological label of claim 5, wherein the optically active dopant is a rare earth, transition metal, actinide or lanthanide.

10. The biological label of claim 5, wherein the optically active active dopant is selected from the group consisting of titanium, vanadium, chromium, iron, cobalt, nickel, zinc, zirconium, niobium, cadmium, hafnium, tantalum, tungsten, rhenium, osmium, iridium, platinum, gold, mercury, cerium, praseodymium, neodymium, promethium, samarium, europium, gadolinium, terbium, dysprosium, holmium, erbium, thulium, ytterbium, and uranium.

11. The biological label of claim 1, wherein the diamondoid is derivatized with a functional group capable of binding to a target analyte.

12. The biological label of claim 1, wherein the functional group is a moiety selected from the group consisting of —H, —F, —Cl, —Br, —I, —OH, —SH, —NH₂, —NHCOCH₃, —NHCHO, —CO₂H, —CO₂R′, —COCl, —CHO, —CH₂OH, ═O, —NO₂, —CH═CH₂, —C≡CH and —C₆H₅, and where R′ is an alkyl group.

13. The biological label of claim 1, wherein the nature of the affinity between the diamondoid and the target analyte is selected from the group consisting of van der Waals attractions, hydrophilic attractions, hydrophobic attractions, ionic bonding, covalent bonding, an electrostatic association, and a magnetic association.

14. The biological label of claim 1, wherein the target analyte is selected from the group consisting of a protein, a sugar, a nucleic acid, an antigen, an antibody, a lipid, a cell, and a subcellular organelle.

15. The biological label of claim 1, wherein the bandgap of the diamondoid-containing material is at least about 2 eV.

16. The biological label of claim 1, wherein the bandgap of the diamondoid-containing material is at least about 3 eV.

17. The biological label of claim 1, wherein the bandgap of the diamondoid-containing material is at least about 4 eV.

18. The biological label of claim 1, wherein the bandgap of the diamondoid-containing material is at least about 5 eV.

19. The biological label of claim 1, further including impurity atoms that contribute electronic states within the bandgap of the diamondoid-containing material.

20. The biological label of claim 5, wherein the diamondoid is derivatized with a functional group capable of binding to a target analyte.

21. The biological label of claim 5, wherein the functional group is a moiety selected from the group consisting of —H, —F, —Cl, —Br, —I, —OH, —SH, —NH₂, —NHCOCH₃, —NHCHO, —CO₂H, —CO₂R′, —COCl, —CHO, —CH₂OH, ═O, —NO₂, —CH═CH₂, —C≡CH and —C₆H₅, and where R′ is an alkyl group.

22. The biological label of claim 5, wherein the nature of the affinity between the diamondoid and the target analyte is selected from the group consisting of van der Waals attractions, hydrophilic attractions, hydrophobic attractions, ionic bonding, covalent bonding, an electrostatic association, and a magnetic association.

23. The biological label of claim 5, wherein the target analyte is selected from the group consisting of a protein, a sugar, a nucleic acid, an antigen, an antibody, a lipid, a cell, and a subcellular organelle.

24. The biological label of claim 5, wherein the bandgap of the diamondoid-containing material is at least about 2 eV.

25. The biological label of claim 5, wherein the bandgap of the diamondoid-containing material is at least about 3 eV.

26. The biological label of claim 5, wherein the bandgap of the diamondoid-containing material is at least about 4 eV.

27. The biological label of claim 5, wherein the bandgap of the diamondoid-containing material is at least about 5 eV.

28. The biological label of claim 5, further including impurity atoms that contribute electronic states within the bandgap of the diamondoid-containing material.

29. A method of detecting a target analyte, the method comprising the steps of:
a) providing a heterodiamondoid-containing probe;
b) binding the heterodiamondoid-containing probe to the target analyte, thus creating a biological label;
c) exciting the biological label with energy such that the biological label is caused to luminesce; and
d) detecting light emitted from the excited biological label.

30. The method of claim 29, wherein the energy is in the form of a beam of photons, such that the luminescent event is photoluminescence.

31. The method of claim 29, wherein the energy is in the form of a beam of electrons, such that the luminescent event is electroluminescence.

32. The method of claim 29, wherein the energy is in the form of heat, such that the luminescent event is thermoluminescence.

33. The method of claim 29, wherein the energy is in the form of chemical energy, such that the luminescent event is chemiluminescence.

34. The method of claim 29, wherein the energy results from the frictional contact between two surfaces, such that the luminescent event is triboluminescence.

35. The method of claim 29, wherein step a) includes substitutionally positioning a nitrogen heteroatom on a diamondoid lattice site adjacent to at least one vacancy or pore.

36. The method of claim 29, further including the step of positioning impurity atoms within the diamondoid-containing material to create electronic states within the bandgap of the diamondoid-containing material.

37. The method of claim 29, further including the step of passing the biological label through a cell membrane after the heterodiamondoid-containing probe is bound to the target analyte.

38. The method of claim 29, further including the step of passing the heterodiamondoid-containing probe through a cell membrane, and then reacting the heterodiamondoid-containing probe with the target analyte.

39. The method of claim 29, wherein the detection of light emitted from the biological label is carried out using a photomultiplier tube.

40. The method of claim 29, wherein the detection of light emitted from the biological label is carried out using a charge-coupled device.
